(19) United States
(12) Patent Application Publication
(10) Pub. No.: US 2007/0298445 A1
(43) Pub. Date: Dec. 27, 2007

(54) Cancer Therapeutic

(76) Inventors: Douglas Boyd, Sugar Land, TX (US); Lin Yang, Houston, TX (US)

Correspondence Address: BAKER BOTTS, LLP, 910 LOUISIANA, HOUSTON, TX, 77002-4995, US

(21) Appl. No.: 11/682,184

(22) Filed: Mar. 5, 2007

Related U.S. Application Data
(60) Provisional application No. 60/779,073, filed on Mar. 3, 2006.

(51) Int. Cl. G01N 33/574; C07H 21/04; C07K 16/18; C12N 5/06

(52) U.S. Cl. 435/7.23; 435/375; 530/387.3; 530/387.9; 530/388.1; 536/24.5

(57) ABSTRACT

Methods for the delivery of a siNA to a cell via a liposome are provided. In certain embodiments, the siNA may bind to a nucleotide sequence encoding ZNF306 protein. These methods may be used to treat a disease, such as cancer. One example of a composition is a composition comprising a siNA component that binds to a nucleotide sequence encoding ZNF306 protein and a lipid component. One example of a method is a method of treating cancer.

### CROSS-REFERENCE TO RELATED APPLICATION

This application claims priority to U.S. Provisional Application Ser. No. 60/779,073 filed on Mar. 3, 2006, the entirety of which is incorporated by reference herein.

### STATEMENT OF GOVERNMENT INTEREST

This disclosure was developed at least in part using funding from the National Institutes of Health Grant numbers #CA58311, #DE10845, and #CA89002. The U.S. government may have certain rights in the invention.

### SEQUENCE LISTING

This disclosure includes a sequence listing submitted as a text file pursuant to 37 C.F.R. §1.52(e)(v) named sequence listing.txt, created on Mar. 5, 2007, with a size of 2,713 bytes, which is incorporated herein by reference. The attached sequence descriptions and Sequence Listing comply with the rules governing nucleotide and/or amino acid sequence disclosures in patent applications as set forth in 37 C.F.R. §§1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984). The symbols and format used for nucleotide and amino acid sequence data comply with the rules set forth in 37 C.F.R. §1.822.

### BACKGROUND

The present disclosure generally relates to delivery of therapeutic compounds. In particular, the present disclosure relates to the delivery of siNA (e.g., a siRNA) via neutral lipid compositions or liposomes and associated methods of use in the treatment of disease.

Sporadic colorectal cancer is one of the most prevalent cancers in industrialized countries and afflicts some 145,000 individuals each year in the United States. Unfortunately, while the prognosis for early staged disease is good, only 5% of those patients with Dukes Stage D survive beyond 5 years (de la Chapelle, A. (2004). Genetic predisposition to colorectal cancer. Nature Reviews 4, 769-780.). Additionally, chemotherapy has provided only an incremental increase in survival in the past 5 years, and patients with metastatic disease have a median survival of ˜20 months. Accordingly, there is a real need to identify genetic events that drive the progression of this disease.

Over the past 20 years, a great deal has been learned regarding the molecular lesions underlying colorectal cancer development. Earlier studies had convincingly demonstrated a contributory role for the adenomatous polyposis coli (APC) gene in the tumorigenic process (Mehlen, P., Fearon, E. R. (2004). Role of the dependence receptor DCC in colorectal cancer pathogenesis. Journal of Clinical Oncology 22, 3420-3428.). In the Wnt pathway, APC binds newly synthesized β-catenin, the latter phosphorylated and degraded by the proteasomal pathway (Radtke, F., Clevers, H. (2005). Self-renewal and cancer of the gut: Two sides of a coin. Science 307, 1904-1909). However, in cancer, the APC gene located on chromosome 5q21 is commonly mutated as a consequence of mutations in the mismatch repair genes Msh2 and Mlh1 (Heyer, J., Yang, K., Lipkin, M., Edelmann, W., Kucherlapati, R. (1999) Mouse models for colorectal cancer. On-cogene 18, 5325-5333.) leading to truncated proteins unable to stimulate the degradation β-catenin. As a consequence, β-catenin accumulates in the nucleus and, in conjunction with Tcf/Lef proteins (Radtke, F., Clevers, H., 2005), activates expression of genes involved in the proliferative response (c-Jun, Fra-1, (Mann, B., Gelos, M., Wiedow, A., Hanski, M. L., Gratchev, A., Ilyas, M., Bodmer, W. F., Moyer, M. P., Riecken, E. O., Buhr, H. J., Hanski, C. (1999). Target genes of b-catenin-T cell factor/lymphoid-enhancer factor signaling in human colorectal carcinomas. Proceedings of the National Academy of Sciences USA 96, 1603-1608.) c-myc, c-myb (Barker, N., Hurlstone, A., Musisi, H., Miles, A., Bienz, M., Clevers, H. (2001). The chromatin remodeling factor Brg-1 interacts with b-catenin to promote target gene activation. European Molecular Biology Organization 20, 4935-4943.). More recently, the Ephrin B (EphB) gene, encoding guidance receptors controlling intestinal epithelial architecture, also in the Wnt pathway, has been implicated as a tumor suppressor in colon carcinogenesis. EphB expression is lost at the adenoma-carcinoma transition and a dominant negative EphB accelerates tumorigenesis in the colon and rectum of APC^{+/Min} mice (Batlle, E., Bacani, J., Begthel, H., Jonkeer, S., Gregorieff, A., van de Born, M., Malats, N., Sancho, E., Boon, E., Pawson, T., Gallinger, S., Pals, S., Clevers, H. (2005). Eph receptor activity suppresses colorectal cancer progression. Nature.).

Earlier studies had also implicated the DCC (deleted in colon cancer) gene located on chromosome 18q21 in the pathogenesis of colon cancer (Mehlen and Fearon, 2004). DCC encodes a transmembrane glycoprotein bound by the netrin-1 ligand (Mehlen and Fearon, 2004). Somatic mutations giving rise to the inclusion of a 120-300 base pair dinucleotide tract in the intron immediately downstream of exon 7 are evident in 10-15% of all colorectal cancers. However, the role of DCC in tumorigenesis is still debatable since heterozygous inactivation of the murine gene does not predispose to cancer (Mehlen and Fearon, 2004), and its chromosomal locus also harbors the MADH4 (encoding the Smad4 transcription factor) the latter that has clearly been implicated in cancer development.

In addition to these genes, mutations in p53 (Calistri, D., Rengucci, C., Seymour, I., Lattuneddu, A., Polifemo, A., Monti, F., Saragoni, L., Amadori, D. (2005). Mutation analysis of p53, K-ras, and BRAF genes in colorectal cancer progression. Journal of Cell Physiology 204, 484-488) and Ki-Ras are present in one third to one half of colorectal cancers (Bos, J. L. (1989). ras oncogenes in human cancer: a review. Cancer Research 49, 4682-4689.; Shirasawa, S., Furuse, M., Yokoyama, N., Sasazuki, T. (1993). Altered growth of human colon cancer cell lines disrupted at activated Ki-ras. Science 260, 85-88.; Smith, G., Carey, F. A., Beattie, J., Wilkie, M. J. V., Lightfoot, T. J., Coxhead, J., Garner, R. C., Steele, R. J. C., Wolf, R. C. (2002). Mutations in APC, Kirsten-ras and p53-alternative genetic pathways to colorectal cancer. Proceedings of the National Academy of Sciences USA 99, 9433-9438; Janssen, K. P., El Marjour, F., Pinto, D., Sastre, X., Rouillard, D., Fouquet, C., Soussi, T., Louvard, D., Robine, S. (2002). Targeted expression of oncogenic K-ras in intestinal epithelium causes spontaneous tumorigenesis in mice. Gastroenterology 132, 492-504.) and these lesions contribute more to the progression of the disease. TGF-β signaling which induces growth arrest by way of the Smad transcription factors is also targeted in colon cancer. The Smads induce expression of CDK inhibitors which in turn interact and interfere with cyclins A, E and D (Arber, N., Doki, Y., Han, E. K. H., Sgambato, A., Zhou, P., Kim, N. H., Klein, M. G., Holt, P. R., Weinstein, I. B. (1997). Antisense to cyclin D1 inhibits the growth and tumorigenicity of human colon cancer cells. Cancer Research 57, 1569-1574.; Derynck, R., Akhurst, R. J., Balmain, A. (2001). TGF-b signaling in tumor suppression and cancer progression. Nature Genetics 29, 117-129.). In colorectal cancer, the TGFBR2 gene, encoding the TGF-β type II receptor, is mutated in up to 25% of all tumors (Derynck et al., 2001). Accordingly, cells harboring this mutation become refractory to the anti-proliferative effects of TGF-β leading to an increased growth fraction. Interestingly, biallelic inactivation of MADH4, the gene encoding Smad4, is often evident in colorectal cancer and the contribution of this inactivation to the disease is clear in genetic models of colon cancer. Thus, while APC^{+/Min} mice only develop adenomas, mice heterozygous for Smad4 and also harboring a mutated APC allele now show invasive adenocarcinoma of the small intestine (Derynck et al., 2001).

Certainly, as described above, much has been learned as to the genetic lesions driving colorectal carcinogenesis and progression so much so that the “Vogelgram” depicting colorectal cancer as the accumulation in the mutations and/or loss of a set of genes including APC, p53, K-Ras has become widely accepted. However, recent studies challenge this dogma for colorectal cancer development. Indeed, less than 7% of colorectal cancers contain simultaneous mutations of APC, K-Ras and p53 and 39% of tumors harbored mutations in only 1 of these genes (Smith et al., 2002). Further 11% of colorectal tumors fail to show simultaneous mutations in any of these three genes (Smith et al., 2002). Moreover, in a recent independent study, Calistri and co-workers (Calistri et al., 2005) in examining 100 colorectal cancers by single strand conformation polymorphism observed a minimal or no copresence of mutations in p53, K-ras and BRAF and noted that mutations of these 3 genes were absent from about one third of the cancers. Together, these studies raise the possibility that the widely accepted genetic model of colorectal cancer development needs to be expanded to accommodate the contribution of other genes.

Indeed, recent studies are now identifying additional genes also contributory to colorectal tumorigenesis arguing for multiple pathways to colorectal cancer development. As one example, allelic imbalance on chromosome 22q has led to the identification of MYO18B as a putative tumor suppressor gene (Nakano, T., Tani, M., Nishioka, M., Kohno, T., Otsuka, A., Ohwada, S., Yokota, J. (2005). Genetic and epigenetic alterations of the candidate tumor-suppressor gene Myo18B, on chromosome arm 22q, in colorectal cancer. Genes, Chromosomes & Cancer 43, 162-171.). Similarly, the RE1-silencing transcription factor (REST), a frequent target of deletion in colorectal cancer as evident in CGH analysis, also likely represents a novel tumor suppressor in this cancer by way of suppressing the PI(3)K signaling pathway (Westbrook, T. F., Martin, E. S., Schlabach, M. R., Leng, Y., Liang, A. C., Feng, B., Zhao, J. J., Roberts, T. M., Mandel, G., Hannon, G. J., Depinho, R. A., Elledge, S. J. (2005). A genetic screen for candidate tumor suppressors identifies REST. Cell 121, 837-848.). Similarly, other recent studies show somatic mutations of genes in signaling pathways (MKK4/JNKK1) (Parsons, D. W., Wang, T.-L., Samuels, Y., Bardelli, A., Cummins, J. M., DeLong, L., Silliman, N., Ptak, J., Szabo, S., Willson, J. K. V., Markowitx, S., Kinzler, K. W., Vogelstein, B., Lengauer, C., Velculescu, V. E. (2005). Mutations in signalling pathways. Nature 436, 792.) as well as in PIK3CA, encoding the p110a catalytic subunit in up to 40% of colorectal cancers (Samuels, Y., Wang, Z., Bardelli, A., Silliman, N., Ptak, J., Szabo, S., Yan, H., Gazdar, A., Powell, S. M., Riggins, G. J., Willson, J. K. V., Markowitx, S., Kinzler, K. W., Vogelstein, B., Velcelescu, V. E. (2004). High frequency of mutations of the PIK3CA gene in human cancers. Science 304, 554.). The contribution of the latter to colon cancer development was likely since generation of a “hot spot” mutation (H1047R) in this kinase increased its activity an event necessary for the transformed phenotype as shown by others (Westbrook et al., 2005).

These aforementioned studies suggest that colorectal cancer can arise by multiple pathways and presumably other, currently unknown, genes also contribute to tumorigenesis. In data-mining of the UniGene Cluster Expression Information (2004 release) database, a transcript encoding a novel zinc finger protein (ZNF306, accession code BC006118) was recognized, whose expression by virtual Northern blotting was highest in colon cancers.

The ZNF306 coding sequence (accession # BT007427) was originally generated as part of a collection of human, full length expression clones. Annotation of the human genome indicated that the corresponding gene maps to chromosome 6p22.1 and is comprised of 6 exons, the first of which is non-coding. The 2.2 kb ZNF306 transcript predicts a 60 kDa protein of 538 amino acids (http://www.ebi.uniprot.org/) with strong characteristics of a transcription factor. Located at the amino-terminal end of the predicted protein sequence is a SCAN domain (amino acids 46-128) (present in many zinc-finger transcription factors) a highly conserved, leucine-rich motif of approximately 60 amino acid (FIG. 1B). The SCAN domain is a protein oligomerization domain whose proposed function, at least based on precedents with other zinc finger proteins, is to recruit trans-activators and co-repressors necessary for transcriptional regulation. A Kruppel-associated box (KRAB), found in about a third of Kruppel-type C2H2 zinc finger proteins, is located 3′ of the SCAN domain (amino acids 214-274). KRAB domains typically function as transcriptional repressors at least when tethered to template DNA. At the carboxy-terminus of the ZNF306 protein are 7 tandem C2H2 zinc fingers (defined by the highly conserved connecting sequence TGEKPYX) well recognized for their role in DNA-binding (Pi, H., Li, Y., Zhu, C., Zhou, L., Luo, K., Yuan, W., Yi, Z., Wang, Y., Wu, X., Liu, M. (2002). A novel human SCAN9Cys)2(His)2 zinc finger transcription factor ZNF232 in early human embryonic development. Biochemical and Biophysical Research Communications 296, 206-213.).

Aside from these genetic factors, vascular endothelial growth factor (VEGF)-mediated angiogenesis is also thought to play a critical role in tumor growth and metastasis. Consequently, anti-VEGF therapies are being actively investigated as potential anti-cancer treatments, either as alternatives or adjuncts to conventional chemo or radiation therapy. Recent evidence from phase III clinical trials led to the approval of bevacizumab, an anti-VEGF monoclonal antibody, by the FDA as first line therapy in metastatic colorectal carcinoma in combination with other chemotherapeutic agents. In addition, there are several ongoing phase III clinical trials using bevacizumab in combination with other chemotherapeutic and anti-angiogenesis agents in the treatment of pancreatic adenocarcinoma, metastatic colorectal carcinoma and advanced renal cell carcinoma. Even more phase II trials are currently ongoing involving the use of combination therapy with bevacizumab to treat advanced or metastatic malignancies, including melanoma, head and neck, breast, lung, ovarian and pancreatic cancer. The efficacy of bevacizumab in treating hematologic malignancies is also being actively investigated. (Cardones A R, Banez L L, Curr Pharm Des. 2006; 12(3): 387-94).

Scientists have discovered a number of agents that inhibit key enzymatic reactions in biochemical pathways that frequently become altered in cancer progression. Antimetabolites are a class of anti-cancer agents that, in general, interfere with normal metabolic pathways, including those necessary for making new DNA. A widely used antimetabolite that thwarts DNA synthesis by interfering with the nucleotide (DNA components) production is 5-fluorouracil. It has a wide range of activity in many cancers including colon cancer, breast cancer, head and neck cancer, pancreatic cancer, gastric cancer, anal cancer, esophageal cancer and hepatomas. Similar to the VEGF inhibitor, bevacizumab, 5-fluorouracil is being actively investigated in combination therapy with several agents in several ongoing clinical trials including, liver cancer, biliary cancer, colon cancer, colorectal cancer, rectal cancer, anal cancer, renal cell carcinoma, bladder cancer, gastric cancer, stomach cancer, esophageal cancer, pancreatic cancer, head and neck cancer, breast cancer, ovarian, endometrial, cervical, non-small cell lung cancer, and neuroendocrine cancer. (http://www.oncolink.com and http://www.clinicaltrials.gov).

### SUMMARY

The present disclosure generally relates to delivery of therapeutic compounds. In particular, the present disclosure relates to the delivery of siNA (e.g., a siRNA) via neutral lipid compositions or liposomes and associated methods of use in the treatment of disease.

Short interfering RNA (siRNA) is well known in the art, but delivery of siRNA in vivo has proven to be very difficult, thus limiting the therapeutic potential of siRNA. Since its description in C. elegans (Fire et al., Nature, 391(6669):806-811, 1998.) and mammalian cells (Elbashir et al., Nature, 411(6836):494-498, 2001.), use of siRNA as a method of gene silencing has rapidly become a powerful tool in protein function delineation, gene discovery, and drug development (Hannon and Rossi, Nature, 431:371-378, 2004). The promise of specific RNA degradation has also generated much excitement for possible use as a therapeutic modality (Ryther et al., Gene Ther., 12(1):5-11, 2004.), but decifering acceptable delivery vehicles has proven difficult.

Delivery methods that are effective for other nucleic acids are not necessarily effective for siRNA (Hassani et al., J. Gene Med., 7(2):198-207, 2005.). Therefore, most studies using siRNA in vivo involve manipulation of gene expression in a cell line prior to introduction into an animal model (Brummelkamp et al., Cancer Cell, 2:243-247, 2002; Yang et al., Oncogene, 22:5694-5701, 2003), or incorporation of siRNA into a viral vector (Xia et al., Nat. Biotechnol., 20:1006-1010, 2002; Devroe and Silver, Expert Opin. Biol. Ther., 4:319-327, 2004). Delivery of “naked” siRNA in vivo has been restricted to site-specific injections or through high-pressure means that are not clinically practical. One study that showed in vivo uptake and targeted downregulation of an endogenous protein by an siRNA after normal systemic dosing required chemical modification of the siRNA (Soutschek et al., Nature, 432:173-178, 2004); however, this chemical modification has an unknown toxicity and may result in significant toxicity to a subject in vivo. Further this chemical modification may affect siRNA activity and/or longevity. The methods and compositions of the present disclosure overcome these limitations of in vivo siRNA delivery.

An aspect of the present disclosure relates to a composition comprising a siNA component and a lipid component, wherein the lipid component has an essentially neutral charge. The lipid component may be in the form of a liposome. The siNA (e.g., an siRNA) may be encapsulated in the liposome. In certain embodiments, the composition may be comprised in a pharmaceutically acceptable carrier. The pharmaceutically acceptable carrier may be formulated for administration to a human. In certain embodiments, the siNA component may bind to a nucleotide sequence encoding ZNF306 protein.

In certain embodiments, the siNA component comprises a single species of siRNA. In other embodiments, the siNA component comprises a two or more species of siRNA. The composition may further comprise a chemotherapeutic. In certain embodiments, the lipid component is in the form of a liposome and the chemotherapeutic is encapsulated within the liposome. In further embodiments, the siNA is a siRNA and the siRNA is encapsulated within the liposome.

In other embodiments, the present disclosure provides an antibody comprising a human constant region that binds to at least a portion of a ZNF306 protein.

Another aspect of the present invention involves a method for delivering a siNA to a cell comprising contacting the cell with the composition. The cell may be comprised in a subject, such as a human. The method may further comprise a method of treating cancer. The cancer may have originated in the bladder, blood, bone, bone marrow, brain, breast, colon, rectum, esophagus, gastrointestine, gum, head, kidney, liver, lung, nasopharynx, neck, prostate, skin, stomach, testis, tongue, or uterus. In certain embodiments, the cancer is ovarian cancer. In certain embodiments, the method further comprises a method of treating a non-cancerous disease. The cell may be a pre-cancerous or a cancerous cell. In certain embodiments, the composition inhibits the growth of the cell, induces apoptosis in the cell, and/or inhibits the translation of an oncogene. The siNA may inhibit the translation of a gene that is overexpressed in the cancerous cell. In certain embodiments, the method further comprises administering an additional therapy to the subject. The additional therapy may comprise administering a chemotherapeutic (e.g., 5-fluorouracil), a surgery, a radiation therapy, and/or a gene therapy.

In other embodiments, the present disclosure provides a method for screening a compound that inhibits or prevents cancer cell proliferation, the method comprising determining a first amount of ZNF306 protein expressed by cancer cells exposed to the compound, wherein the cancer cells overexpress ZNF306 protein; and comparing the first amount of ZNF306 protein to a second amount of ZNF306 protein expressed by the cancer cells that have not been exposed to the compound; whereby the first amount being less than the second amount indicates that the compound may inhibit or prevent ZNF306 cancer cell proliferation.

In certain other embodiments, the present disclosure further provides a method of preventing growth of a cancerous or precancerous mammalian cell comprising administering to the cell a composition a siNA component that binds to a nucleotide sequence encoding ZNF306 protein and a lipid component, wherein the siNA prevents translation of a gene transcript that promotes growth of the cancerous or precancerous mammalian cell.

In other embodiments, the present disclosure provides a method of treating cancer comprising administering to a mammal a composition comprising a siNA component that binds to a nucleotide sequence encoding ZNF306 protein and a lipid component.

The features and advantages of the present invention will be readily apparent to those skilled in the art upon a reading of the description of the embodiments that follows.

### DRAWINGS

Some specific example embodiments of the disclosure may be understood by referring, in part, to the following description and the accompanying drawings.

FIGS. 1A-B show that Unigene Cluster Expression reveals elevated ZNF306 transcript levels in colon tumors. FIG. 1A shows normalized ZNF306 expression in different tissues. Data indicates relative expression of ZNF306 in different tissues normalized for the number of clones from each tissue included in the Unigene database (2004 release). FIG. 1B demonstrates a schematic of the various predicted domains in the ZNF306 protein.

FIG. 2: Semi-quantitation of ZNF306 mRNA levels in resected colon cancers. Total RNA was prepared from frozen colon tissue (50 mg) by homogenization in 1 ml of TRIZOL Reagent. RNA (20 μg) was treated with 40 mU/μl TURBO DNA-free DNase enzyme. After DNase inactivation, 2 μg of RNA was reverse transcribed with AMV Reverse Transcriptase. Multiplex PCR was performed with 100 ng each of the following ZNF306 primers (5′-GGC CCT GAC CCT CAC CCC-3′ and 5′-CAG ATG TGC CGC CTC CCT CC-3′ spanning exons 5 and 6), β-actin primers (10 ng) and 1U Taq polymerase using 30 cycles. PCR products were visualized by staining with ethidium bromide. T, tumor; N— non-malignant adjacent mucosa.

FIGS. 3A-D shows elevated ZNF306 mRNA amounts in poorly differentiated colorectal cancers. FIG. 3A demonstrates the morphology of the indicated cells stained with Hema Diff. FIG. 3B shows the semi-quantitation of ZNF306 mRNA levels by RT-PCR as described in the legend to FIG. 2. FIG. 3C demonstrates the real-time quantitative PCR measuring ZNF306 mRNA levels using SYBR Green and primers as described in the legend to FIG. 2. FIG. 3D depicts the melting curve showing a single amplified product generated in the real-time PCR.

FIGS. 4A-E show ZNF306 over-expression increases colon cancer growth in semi-solid medium. N-terminus-flag-tagged ZNF306 was sub-cloned into the pIRES2-EGFP bicistronic vector (FIG. 4A) and HCT116 cells transfected with this Flag-tagged ZNF306 expression construct. Cells were selected with 1 mg/ml G418 and after 2 weeks, a G418-resistant GFP-positive clone (FIG. 4B) was harvested, and analyzed for ZNF306 expression (FIG. 4C) using the anti-Flag M2 antibody. FIG. 4D and FIG. 4E The indicated cells (80,000) were grown in 0.35% agar and the colonies visualized and enumerated after 14 days. The data represent average colony #+SD (from 5 independent fields).

FIGS. 5A-D illustrate virally transduced ZNF306 increased colon cancer growth in semi-solid medium. FIG. 5A shows that the Flag-tagged ZNF306 coding sequence was subcloned into the pLAPSN retroviral vector deleted of the alkaline phosphatase gene. 293 packaging cells were transiently transfected with this flag-tagged ZNF306 expression construct using Lipofectamine 2000. Viral supernatant collected at 12 h intervals for up to 48 h post-transfection was filtered and used to transduce HCT116 cells using polybrene (final concentration=4 μg/ml). FIG. 5B demonstrates that after 48 h, cells were harvested and analyzed for ZNF306 mRNA by RT-PCR. FIGS. 5C-5D shows that colony growth in soft agar was assessed as described in the legend to FIG. 4.

FIGS. 6A-B: Exogenous ZNF306 expression renders colon cancer cells resistant to anoikis. Parental HCT116 cells (50,000) or clones expressing the empty vector or the ZNF306 cDNA were cultured in plates coated with a hydrogel layer that hinders cell attachment. After 2 days, cells were dispersed with trypsin and then subjected to FACS analysis (FIG. 6A) after staining with propidium iodide. The % of apoptotic cells (FIG. 6B) corresponding to cells in the sub-G1 population is shown. The HCT116 ZNF306 column represents the average from both clones.

FIGS. 7A-D show exogenous ZNF306 expression increased tumorigenesis in vivo. The indicated cells were harvested and suspended in HBSS and Trypan Blue exclusion performed to confirm viability in excess of 95%. Cells (106) in 50 μl of HBSS were injected intracecally. After 7 weeks, mice were sacrificed and tumors (FIGS. 7A & 7B) harvested, weighed (FIG. 7D) and sections H&E stained and examined histologically (FIG. 7B—N represents the normal colonic crypt and T indicates tumor). FIG. 7C-analysis, as described in the legend to FIG. 2, of ZNF306 expression in the indicated tumors by RT-PCR.

FIGS. 8A-B show siRNA-targeting of the ZNF306 transcript reduces colony formation. The optimal target sequence (determined by the Oligoengine Workstation 2) for ZNF306 (UAUCGUGCCACCUGAGAGA) or the scrambled sequence (Control), was cloned into pSUPERIOR.retro.puro vector. 293 packaging cells were transfected with pSUPERIOR.retro.puro vector encoding these sequences and the resulting retrovirus used to transduce HCT116. Cells were selected with puromycin and analyzed by RT-PCR to detect ZNF306 expression (FIG. 8A) or grown in soft agar for the specified times (FIG. 8B) as described in the legend to FIG. 4.

FIG. 9: Sub-cellular localization of ZNF306. RKO colon cancer cells were transiently tranfected with the pcDNA3-Flag-ZNF306 expression vector. After 48 h, cells were subjected to immunofluorescence with the anti-Flag antibody (1:400 dilution) and an FITC-conjugated secondary antibody and counterstained with DAPI to localize nuclei.

FIG. 10A-D illustrate CAST-ing to identify a consensus DNA-binding sequence for ZNF306. FIG. 10A shows a schematic of the CAST-ing method. Lystate from HCT116 cells stably expressing ZNF306 was purified with an anti-Flag M2 affinity resin and subsequently eluted with a 3× tandem-repeated Flag peptide (FIG. 10B Lane 1) and visualized by Western blotting. FIGS. 10C-10E. A random oligonucleotide library (500 ng) (CACGTGAGTTCAGCGGATCCTGTCGNNNNNNNNNNNNNNNNNNNNNNNNNNGAGGCGAATTCAGTGCAACTGCAGC-3′) was incubated with 10 μl of the resin-immobilized Flag-ZNF306 protein in the presence of 2 μg poly dI.dC and 10 μg acetylated BSA. DNA was phenolchloroform extracted, precipitated and amplified by 15 PCR cycles (using primers to the arms of the oligonucleotides) to enrich the ZNF306-bound oligonucleotides. The amplified PCR products were purified and the process repeated 6 times (FIGS. 10A, 10C). In the final round, DNA was labeled with radioactive dCTP and subjected to EMSA (FIG. 10D) using a range (1-100 ng) of purified Flag-tagged ZNF306 protein.

FIG. 11 is a chart illustrating several transcripts up-regulated in ZNF306 over-expression colon cancer tumors identified by expression profiling. Total RNA was prepared from tumors generated orthotopically (see FIG. 7) and analyzed for differentially expressed transcripts using the U133A 2.0 Affymetrix chip which harbors cDNAs to ˜18,400 mRNAs. The fold induction represents the signal generated with tumors generated with ZNF306-overexpressing HCT116 cells as a function of the signal generated with tumors from the vector-bearing cells.

FIGS. 12A-B illustrates a predicted hydrophobicity plot for ZNF306 and peptide selection for generation of an anti-ZNF306 antibody. FIG. 12A shows the ZNF306 amino acid sequence. Table 4 below indicates the abbreviations for amino acids as used in FIG. 12A. FIG. 12B shows a hydrophobicity plot for the ZNF306 protein as analyzed by the Kyte-Doolittle Hydropathy algorithm thus generating 3 potential antigenic peptides. Of these 3 peptides only one (bold type) was deemed to be unique after a BLAST search and was therefore selected as immunogen.

FIG. 13 shows HT29 transduced with siRNA ZNF306 (bottom) or vector only [pSUPER] (top). Cells were selected with puromycin (6 μg/ml) for 1 week. Resistant cells (5,000) were analyzed for growth in soft agar. Photomicrographs are taken 2 weeks later.

FIG. 14 shows HCT116 cells expressing empty vector or ZNF306 cDNA were treated with the indicated 5-fluorouracil concentrations. Viable cells were counted 6 days later.

FIG. 15A shows HCT116 or PC3 cells expressing an empty vector or the ZNF306 Coding sequence were lysed and subjected to Western blotting using a 1:10,000 dilution of the anti-serum generated against a KLH-coupled peptide (EGRERFRGFRYPE) derived from the predicted ZNF306 protein sequence. FIG. 15B demonstrates the same as FIG. 15A with the exception that 4 parental colon cancer cell lines were compared for endogenous ZNF306 protein. Note that the exposure in FIG. 15B is longer than FIG. 15A to reveal the endogenous protein.

FIG. 16 illustrates immunohistochemistry showing reactivity (brown color) most pronounced in the tumor. A 1:2000 dilution of the ZNF306 antiserum was used. DAB was used to visualize immunoreactivity.

FIG. 17 illustrates the distinction between Stage II and Stage IV tissue arrays.

FIG. 18 illustrates the results of immunohistochemistry on colorectal tissue microarray of stage IV and II tissues.

FIGS. 19A-H illustrates that ZNF306 knockdown modulates colon cancer tumorigenecity. FIG. 19A illustrates the results of analysis by RT-PCR of ZNF306 mRNA levels for RKO colon cancer cells after transduction with a retro-virus encoding a ZNF306 targeting shRNA or the scrambled sequence. FIG. 19B shows results of Western Blotting. FIGS. 19C and D shows the results of analysis for growth in soft agar, illustrating that ZNF306 repression markedly reduced anchorage-independent growth. FIG. 19E shows the results of an MTT assay, indicating that reduction in colony number unlikely reflected slower monolayer proliferation. FIGS. 19F and G illustrate the presence of dramatically smaller tumors in mice intracecally injected with RKO cells knocked down for ZNF306 compared RKO cells transduced to express scrambled shRNA. FIG. 19H shows the results of RT-PCR confirming ZNF306 transcript knockdown in pooled tumor tissue from mice injected with ZNF306-silencing vectors.

FIGS. 20A-C illustrate that ZNF306 does not stimulate p53, Tcf/Lef and TGF-β responsive reporters. FIG. 20A illustrates that in RKO cells, wild type for APC and β-catenin , ZNF306 failed to activate the Wnt-responsive TOP flash reporters, whereas the positive control, β-catenin, caused robust induction. FIG. 20B shows ZNF306 did not stimulate TGF-β responsive reporter but successfully activated an artificial promotor. FIG. 20C illustrates that ZNF306 expression had minimal effect on p53 reporter in p53 wt RKO cells.

FIG. 21 shows the results of immunohistochemical detection of ZNF306 protein and β-catenin. Five genetically characterized wild-type colon tumors, in which 100% (5 of 5) showed ZNF306 positive staining; while 20% (1 of 5) was β-catenin negative, 60% (3 of 5) was β-catenin membrane or cytoplasm staining, and 20% (1 of 5) showed a few tumor cells with nuclear staining.

FIGS. 22A-F show that integrin β4 is a downstream effector of ZNF306. FIG. 22A shows RT-PCR results illustrating elevated integrin β4 mRNA in pooled tumors generated with ZNF306-overexpressing HCT116 cells. FIG. 22B shows analysis by Western Blotting of HCT116 cells bearing the empty vector or a corresponding pool of ZNF306 expressing clones, showing increased phosphorylated Akt levels, indicative of activated PI3K signaling. FIG. 22C shows the results of electrophoretic mobility shift assay. FIG. 22D illustrates a schematic of the integrin β4 gene indicating the primers used for chromatin immunoprecipitation. FIG. 22E shows the results of a chromatin immunoprecipitation assay. FIG. 22F is a graph comparing luciferase activity of the ZNF306 expression plasmid and the empty vector. FIG. 22G shows RT-PCR results of HCT116 cells expressing a ZNF306 cDNA or the empty vector after transduction with a retrovirus bearing a integrin-β4 targeting shRNA. Integrin-β4 targeting shRNA ablated the integrin β4 transcript levels in both HCT116 cells expressing ZNF306 and the empty vector. FIG. 22H shows that integrin β4 knockdown countered the ZNF306-dependent augmentation of anchorage-independent growth.

FIG. 23 shows liposomal delivery of siRNA targeting ZNF306 has an in vivo-effect on tumor growth

FIG. 24 shows that large tumors in mice treated with siRNA reflect inefficient knockdown of ZNF306 mRNA.

FIG. 25 shows that liposomal-siRNA also inhibited RKO orthotopic tumor growth.

FIG. 26 shows fluorescent liposome-siRNA compositions indicated the presence of siRNA in tumor cells.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure provides compositions and methods for delivery of a siRNA to a cell via a non-charged liposome. Non-charged liposomes may be used to efficiently deliver a siNA (e.g., an siRNA) to cells in vivo. These methods may be used to treat a cancer.

I. Lipid Preparations

The present disclosure provides methods and compositions for associating a siNA (e.g., a siRNA) with a lipid and/or liposome. The siNA may be encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The liposome or liposome/siNA associated compositions of the present disclosure are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. One example of a lipid includes, but is not limited to, dioleoylphosphatidylcholine (DOPC).

“Liposome” is a generic term encompassing a variety of unilamellar, multilamellar, and multivesicular lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver Diseases, Targeted Diagnosis and Therapy Using Specific Receptors and Ligands, Wu et al. (Eds.), Marcel Dekker, NY, 87-104, 1991.). However, the present disclosure also encompasses compositions that have different structures in solution than the normal vesicular structure. For example, the lipids may assume a micellar structure or merely exist as non-uniform aggregates of lipid molecules. Also contemplated are lipofectamine-nucleic acid complexes.

Liposomes have been used previously for drug delivery (e.g., delivery of a chemotherapeutic). Liposomes (e.g., cationic liposomes) are described in WO02/100435A1, U.S Pat. No. 5,962,016, U.S. Application 2004/0208921, WO03/015757A1, WO04029213A2, U.S. Pat. No. 5,030,453, and U.S. Pat. No. 6,680,068, all of which are hereby incorporated by reference in their entirety without disclaimer. A process of making liposomes is also described in WO04/002453A1. Neutral lipids have been incorporated into cationic liposomes (e.g., Farhood et al., Biochim. Biophys. Act, 289-295, 1995). Liposome-mediated polynucleotide delivery and expression of foreign DNA in vitro has been very successful. Wong et al. (1980) (Wong et al., Gene, 10:87-94, 1980.) demonstrated the feasibility of liposome-mediated delivery and expression of foreign DNA in cultured chick embryo, HeLa and hepatoma cells. Nicolau et al. (1987) (Nicolau et al., Methods Enzymol., 149:157-176, 1987.) accomplished successful liposome-mediated gene transfer in rats after intravenous injection.

In certain embodiments of the present disclosure, the lipid may be associated with a hemaglutinating virus (HVJ). This has been shown to facilitate fusion with the cell membrane and promote cell entry of liposome-encapsulated DNA (Kaneda et al., Science, 243:375-378, 1989). In other embodiments, the lipid may be complexed or employed in conjunction with nuclear non-histone chromosomal proteins (HMG-1) (Kato et al, J. Biol. Chem., 266:3361 3364, 1991). In yet further embodiments, the lipid may be complexed or employed in conjunction with both HVJ and HMG-1. In that such expression vectors have been successfully employed in transfer of a polynucleotide in vitro and in vivo, then they are applicable for the methods and compositions of the present disclosure.

A. Neutral Liposomes

“Neutral liposomes” or “non-charged liposomes”, as used herein, generally refer to liposomes having one or more lipid components that yield an essentially-neutral, net charge (substantially non-charged). The terms “essentially-neutral” or “substantially non-charged”, refer to few, if any, lipid components within a given population (e.g., a population of liposomes) include a charge that is not canceled by an opposite charge of another component (e.g., fewer than 10% of components include a non-canceled charge, more preferably fewer than 5%, and most preferably fewer than 1%). In certain embodiments of the present disclosure, compositions may be prepared wherein the lipid component of the composition is essentially neutral but is not in the form of liposomes.

In certain embodiments, neutral liposomes may include mostly lipids and/or phospholipids that are themselves neutral. In certain embodiments, amphipathic lipids may be incorporated into or used to generate neutral liposomes. For example, a neutral liposome may be generated by combining positively and negatively charged lipids so that those charges substantially cancel one another. For such a liposome, few, if any, charged lipids are present whose charge is not canceled by an oppositely-charged lipid (e.g., fewer than 10% of charged lipids have a charge that is not canceled, more preferably fewer than 5%, and most preferably fewer than 1%). It is also recognized that the above approach may be used to generate a neutral lipid composition wherein the lipid component of the composition is not in the form of liposomes.

In certain embodiments, a neutral liposome may be used to deliver a siRNA. The neutral liposome may contain a siRNA directed to the suppression of translation of a single gene, or the neutral liposome may contain multiple siRNA that are directed to the suppression of translation of multiple genes. Further the neutral liposome may also contain a chemotherapeutic in addition to the siRNA; thus, in certain embodiments, chemotherapeutic and a siRNA may be delivered to a cell (e.g., a cancerous cell in a human subject) in the same liposome. An advantage to using neutral liposomes is that, in contrast to the toxicity that has been observed in response to cationic liposomes, little to no toxicity has yet been observed as a result of neutral liposomes.

In certain embodiments, the lipid component has an essentially neutral charge because it comprises a positively charged lipid and a negatively charged lipid. The lipid component may further comprise a neutrally charged lipid. The neutrally charged lipid may be a phospholipid. The positively charged lipid may be a positively charged phospholipid. The negatively charged lipid may be a negatively charged phospholipid. The negatively charged phospholipid may be a phosphatidylserine, such as dimyristoyl phosphatidylserine (“DMPS”), dipalmitoyl phosphatidylserine (“DPPS”), or brain phosphatidylserine (“BPS”). The negatively charged phospholipid may be a phosphatidylglycerol, such as dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), or dioleoylphosphatidylglycerol (“DOPG”). In certain embodiments, the composition further comprises cholesterol or polyethyleneglycol (PEG). In certain embodiments, the phospholipid is a naturally-occurring phospholipid. In other embodiments, the phospholipid is a synthetic phospholipid.

B. Phospholipids

Liposomes of the present disclosure may comprise a phospholipid. In certain embodiments, a single kind of phospholipid may be used in the creation of liposomes (e.g., DOPC used to generate neutral liposomes). In other embodiments, more than one kind of phospholipid may be used to create liposomes.

Phospholipids include glycerophospholipids and certain sphingolipids. Phospholipids may include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

Phospholipids include, for example, phosphatidylcholines, phosphatidylglycerols, and phosphatidylethanolamines; because phosphatidylethanolamines and phosphatidyl cholines are non-charged under physiological conditions (at about pH 7), these compounds may be particularly useful for generating neutral liposomes. In certain embodiments, the phospholipid DOPC is used to produce non-charged liposomes. In certain embodiments, a lipid that is not a phospholipid may (e.g., a cholesterol) be used

Phospholipids may be from natural or synthetic sources. However, phospholipids from natural sources, such as egg or soybean phosphatidylcholine, brain phosphatidic acid, brain or plant phosphatidylinositol, heart cardiolipin and plant or bacterial phosphatidylethanolamine are not used, in certain embodiments, as the primary phosphatide (i.e., constituting 50% or more of the total phosphatide composition) because this may result in instability and leakiness of the resulting liposomes.

C. Production of Liposomes

Liposomes used according to the present disclosure can be made by different methods. For example, a nucleotide may be encapsulated in a neutral liposome using a method involving ethanol and calcium (Bailey and Sullivan, 2000). The size of the liposomes varies depending on the method of synthesis. A liposome suspended in an aqueous solution is generally in the shape of a spherical vesicle, and may have one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as, for example, the nature of the solvent and the presence of other compounds in the solution.

Lipids suitable for use according to the present disclosure can be obtained from commercial sources. For example, dimyristyl phosphatidylcholine (“DMPC”) may be obtained from Sigma Chemical Co., dicetyl phosphate (“DCP”) may be obtained from K & K Laboratories (Plainview, N.Y.); cholesterol (“Chol”) may be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol may be stored at about −20° C. Chloroform may be used as the only solvent since it is more readily evaporated than methanol.

Liposomes within the scope of the present disclosure may be prepared in accordance with known laboratory techniques. In certain embodiments, liposomes may be prepared by mixing liposomal lipids, in a solvent in a container (e.g., a glass, pear-shaped flask). The container will typically have a volume ten-times greater than the volume of the expected suspension of liposomes. Using a rotary evaporator, the solvent may be removed at approximately 40° C. under negative pressure. The solvent may be removed within about 5 min. to 2 hours, depending on the desired volume of the liposomes. The composition may be dried further in a desiccator under vacuum. The dried lipids generally are discarded after about 1 week because of a tendency to deteriorate with time. Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

Liposomes can also be prepared in accordance with other known laboratory procedures: the method of Bangham et al. (1965) (Bangham et al., J. Mol. Biol., 13(1):253-259, 1965), the contents of which are incorporated herein by reference; the method of Gregoriadis, as described in DRUG CARRIERS IN BIOLOGY AND MEDICINE (1979), the contents of which are incorporated herein by reference; the method of Deamer and Uster (1983) (Deamer and Uster, In: Liposome Preparation: Methods and Mechanisms, Ostro (Ed.), Liposomes, 1983), the contents of which are incorporated by reference; and the reverse-phase evaporation method as described by Szoka and Papahadjopoulos (1978) (Szoka and Papahadjopoulos, Proc. Natl. Acad. Sci. USA, 75:4194 4198, 1978). The aforementioned methods differ in their respective abilities to entrap aqueous material and their respective aqueous space-to-lipid ratios.

Dried lipids or lyophilized liposomes (e.g., prepared as described above) may be dehydrated and reconstituted in a solution of inhibitory peptide and diluted to an appropriate concentration with an suitable solvent (e.g., DPBS). The mixture may then be vigorously shaken in a vortex mixer. Unencapsulated nucleic acid may be removed by centrifugation at 29,000 g and the liposomal pellets washed. The washed liposomes may be resuspended at an appropriate total phospholipid concentration (e.g., about 50-200 mM). The amount of nucleic acid encapsulated can be determined in accordance with standard methods. After determination of the amount of nucleic acid encapsulated in the liposome preparation, the liposomes may be diluted to appropriate concentrations and stored at 4° C. until use.

II. Short Inhibitory Nucleic Acids (siNA)

The term “siNA”, as used herein, refers to a short interfering nucleic acid. Examples of siNA include but are not limited to RNAi, double-stranded RNA, and siRNA. A siNA may inhibit the transcription of a gene in a cell. A siNA may be from 16 to 1000 or more nucleotides long, and in certain embodiments from 18 to 100 nucleotides long. In certain embodiments, the siNA may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. The siNA may comprise a nucleic acid and/or a nucleic acid analog. Typically, a siNA may inhibit the translation of a single gene within a cell; however, in certain embodiments, a siNA may inhibit the translation of more than one gene within a cell. In certain embodiments, the siNA inhibits the translation of a gene that promotes growth of a cancerous or pre-cancerous mammalian cell (e.g., a human cell). The siNA may induce apoptosis in the cell, and/or inhibit the translation of an oncogene. In certain embodiments, the siNA may bind to a nucleotide sequence encoding ZNF306 protein.

Within a siNA, a nucleic acids do not have to be of the same type (e.g., a siNA may comprise a nucleotide and a nucleic acid analog). In certain embodiments, siNA may form a double-stranded structure; the double-stranded structure may result from two separate nucleic acids that are partially or completely complementary. In certain other embodiments the present disclosure, the siNA may comprise only a single nucleic acid or nucleic acid analog and form a double-stranded structure by complementing with itself (e.g., forming a hairpin loop). The double-stranded structure of the siNA may comprise 16 to 500 or more contiguous nucleobases. The siNA may comprise 17 to 35 contiguous nucleobases, more preferably 18 to 30 contiguous nucleobases, more preferably 19 to 25 nucleobases, more preferably 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

siNA (e.g., siRNA) are well known in the art. For example, siRNA and double-stranded RNA have been described in U.S. Pat. Nos. 6,506,559 and 6,573,099, as well as in U.S. Applications 2003/0051263, 2003/0055020, 2004/0265839, 2002/0168707, 2003/0159161, 2004/0064842, all of which are herein incorporated by reference in their entirety.

A. Nucleic Acids

The present disclosure provides methods and compositions for the delivery of siNA via neutral liposomes. Because a siNA is composed of a nucleic acid, methods relating to nucleic acids (e.g., production of a nucleic acid, modification of a nucleic acid, etc.) may also be used with regard to a siNA.

The term “nucleic acid” is well known in the art. A “nucleic acid” as used herein generally refers to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between 3 and about 100 nucleobases in length. The term “polynucleotide” refers to at least one molecule of greater than about 100 nucleobases in length.

These definitions refer to a single-stranded or double-stranded nucleic acid molecule. Double stranded nucleic acids are formed by fully complementary binding, although in some embodiments a double stranded nucleic acid may formed by partial or substantial complementary binding. Thus, a nucleic acid may encompass a double-stranded molecule that comprises one or more complementary strand(s) or “complement(s)” of a particular sequence, typically comprising a molecule. As used herein, a single stranded nucleic acid may be denoted by the prefix “ss” and a double stranded nucleic acid by the prefix “ds”.

1. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, carboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table non-limiting, purine and pyrimidine derivatives and analogs is also provided in Table 1 below.

TABLE 1
Purine and Pyrimidine Derivatives or Analogs

| Abbr. | Modified base description |
|---|---|
| ac4c | 4-acetylcytidine |
| Chm5u | 5-(carboxyhydroxylmethyl) |
| | uridine |
| Cm | 2′-O-methylcytidine |
| Cmnm5s2u | 5-carboxymethylamino-methyl-2- |
| | thioridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2- |
| | methylthiopurine-6-yl)carbamoyl)threonine |
| Mt6a | N-((9-beta-D-ribofuranosylpurine-6-yl)N- |
| | methyl-carbamoyl)threonine |
| Mv | Uridine-5-oxyacetic acid |
| | methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6- |
| | yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, |
| | (acp3)u |

A nucleobase may be comprised in a nucleside or nucleotide, using any chemical or natural synthesis method described herein or known to one of ordinary skill in the art.

2. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, DNA Replication, 2nd Ed., Freeman, San Francisco, 1992).

3. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

4. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, In: Synthesis and Biological Function, Wiley-Interscience, NY, 171-172, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or hinder expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance, cellular uptake and regulating RNA expression; U.S. Pat. No. 5,858,988 which describes hydrophobic carrier agent attached to the 2′-O position of oligonuceotides to enhanced their membrane permeability and stability; U.S. Pat. No. 5,214,136 which describes olignucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

5. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., Nature, 365(6446):566-568, 1993; PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogs such as a peptide nucleic acid may be used to inhibit nucleic acid amplification, such as in PCR™, to reduce false positives and discriminate between single base mutants, as described in U.S. Pat. No. 5,891,625. Other modifications and uses of nucleic acid analogs are known in the art, and it is anticipated that these techniques and types of nucleic acid analogs may be used with the present disclosure. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. U.S. application Ser. No. 117,363 describes several alkylamino moeities used to enhance cellular uptake of a PNA. Another example is described in U.S. Pat. No. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

6. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., Nucleic Acids Res., 14(13):5399-5407, 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., In: Molecular cloning, Cold Spring Harbor Laboratory Press, Cold Spring Harbor, N.Y., 2001, incorporated herein by reference).

7. Purification of Nucleic Acids

A nucleic acid may be purified on polyacrylamide gels, cesium chloride centrifugation gradients, or by any other means known to one of ordinary skill in the art (see for example, Sambrook et al., 2001, incorporated herein by reference).

In certain embodiments, the present disclosure concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

8. Hybridization

As used herein, the term “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture. It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

III. Cancer

The present disclosure may be used to treat a disease, such as cancer. For example, a siRNA may be delivered via a non-charged liposome to treat a cancer. The cancer may be a solid tumor, metastatic cancer, or non-metastatic cancer. In certain embodiments, the cancer may originate in the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In certain embodiments, the cancer is human ovarian cancer. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present disclosure may also be used to treat a non-cancerous disease (e.g., a fungal infection, a bacterial infection, a viral infection, and/or a neurodegenerative disease).

IV. Pharmaceutical Preparations

Where clinical application of non-charged lipid component (e.g., in the form of a liposome) containing a siNA is undertaken, it will generally be beneficial to prepare the lipid complex as a pharmaceutical composition appropriate for the intended application. This will typically entail preparing a pharmaceutical composition that is essentially free of pyrogens, as well as any other impurities that could be harmful to humans or animals. One may also employ appropriate buffers to render the complex stable and allow for uptake by target cells.

The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one non-charged lipid component comprising a siNA or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). A pharmaceutically acceptable carrier is preferably formulated for administration to a human, although in certain embodiments it may be desirable to use a pharmaceutically acceptable carrier that is formulated for administration to a non-human animal but which would not be acceptable (e.g., due to governmental regulations) for administration to a human. Except insofar as any conventional carrier is incompatible with the active ingredient, its use in the therapeutic or pharmaceutical compositions is contemplated.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to mitigate the growth of microorganisms.

The therapeutic compositions of the present disclosure are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents may include, but are not limited to, propylene glycol, polyethylene glycol, vegetable oil and injectable organic esters such as ethyloleate. Aqueous carriers may include, but are not limited to water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

The therapeutic compositions of the present disclosure may include classic pharmaceutical preparations. Administration of therapeutic compositions according to the present invention will be via any common route so long as the target tissue is available via that route. This includes oral, nasal, buccal, rectal, vaginal or topical. Topical administration may be particularly advantageous for the treatment of skin cancers, to mitigate chemotherapy-induced alopecia or other dermal hyperproliferative disorder. Alternatively, administration may be by orthotopic, intradermal subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, the preferred route is aerosol delivery to the lung. Volume of the aerosol is between about 0.01 ml and 0.5 ml. Similarly, a preferred method for treatment of colon-associated disease would be via enema.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

As used herein “prevent” shall mean the inhibition of gene transcript translation and/or the increase of gene transcript degradation.

V. Antibodies

The present disclosure contemplates antibodies having a human constant region that binds to at least a portion of a ZNF306 protein. These antibodies may comprise a complete antibody molecule, having full length heavy and light chains; a fragment thereof, such as a Fab, Fab′, (Fab′)₂, or Fv fragment; a single chain antibody fragment, e.g. a single chain Fv, a light chain or heavy chain monomer or dimer; multivalent monospecific antigen binding proteins comprising two, three, four or more antibodies or fragments thereof bound to each other by a connecting structure; or a fragment or analogue of any of these or any other molecule with the same or similar specificity. A peptide sequence that may be determined based on its hydrophilicity and its sequence as determined by a BLAST search, produced recombinantly or by chemical synthesis, and fragments or other derivatives, may be used as an immunogen to generate the antibodies that recognize the ZNF306 protein, or portions thereof.

“Antibody” as used herein includes polypeptide molecules comprising heavy and/or light chains which have immunoreactive activity. Antibodies include immunoglobulins which are the product of B cells and variants thereof, as well as the T cell receptor (TcR) which is the product of T cells and variants thereof. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Subclasses of heavy chains are also known. For example, IgG heavy chains in humans can be any of IgG1, IgG2, IgG3, and IgG4 subclasses. Immunoglobulins or antibodies can exist in monomeric or polymeric form, for example, IgM antibodies which exist in pentameric form and/or IgA antibodies which exist in monomeric, dimeric or multimeric form.

A typical immunoglobulin structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. The amino acids of an antibody may be naturally or nonnaturally occurring.

Antibodies that contain two combining sites are bivalent in that they have two complementarity or antigen recognition sites. A typical natural bivalent antibody is an IgG. Although vertebrate antibodies generally comprise two heavy chains and two light chains, heavy chain only antibodies are also known. See Muyldermans et al., Trends in Biochem. Sci. 26(4):230-235 (1991). Such antibodies are bivalent and are formed by the pairing of heavy chains. Antibodies may also be multivalent, as in the case of dimeric forms of IgA and the pentameric IgM molecule. Antibodies also include hybrid antibodies wherein the antibody chains are separately homologous with referenced mammalian antibody chains. One pair of heavy and light chain has a combining site specific to one antigen and the other pair of heavy and light chains has a combining site specific to a different antigen. Such antibodies are referred to as bispecific because they are able to bind two different antigens at the same time. Antibodies may also be univalent, such as, for example, in the case of Fab or Fab′ fragments.

Antibodies exist as full length intact antibodies or as a number of well-characterized fragments produced by digestion with various peptidases or chemicals. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding).

Thus, for example, pepsin digests an antibody below the disulfide linkages in the hinge region to produce F(ab)₂, a dimer of Fab which itself is a light chain joined to V_{H}-CH1 by a disulfide bond. F(ab)₂ may be reduced under mild conditions to break the disulfide linkage in the hinge region, thereby converting the F(ab)₂ dimer into a Fab′ monomer. The Fab′ monomer is essentially a Fab fragment with part of the hinge region (see, e.g., Fundamental Immunology (W. E. Paul, ed.), Raven Press, N.Y. (1993) for a more detailed description of other antibody fragments). As another example, partial digestion with papain can yield a monovalent Fab/c fragment. See M. J. Glennie et al., Nature 295:712-714 (1982). While various antibody fragments are defined in terms of the digestion of an intact antibody, one of skill in the art will appreciate that any of a variety of antibody fragments may be synthesized de novo either chemically or by utilizing recombinant DNA methodology. Thus, the term antibody as used herein also includes antibody fragments produced by the modification of whole antibodies, synthesized de novo, or obtained from recombinant DNA methodologies. One skilled in the art will recognize that there are circumstances in which it is advantageous to use antibody fragments rather than whole antibodies. For example, the smaller size of the antibody fragments allows for rapid clearance and may lead to improved access to solid tumors.

Binding fragments are produced by recombinant DNA techniques, or by enzymatic or chemical cleavage of intact immunoglobulins. Binding fragments include Fab, Fab′, F(ab′)₂, Fabc, Fv, single chains, and single-chain antibodies. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

Recombinant antibodies may be conventional full length antibodies, hybrid antibodies, heavy chain antibodies, antibody fragments known from proteolytic digestion, antibody fragments such as Fv or single chain Fv (scFv), single domain fragments such as V_{H} or V_{L}, diabodies, domain deleted antibodies, minibodies, and the like. An Fv antibody is about 50 kD in size and comprises the variable regions of the light and heavy chain. The light and heavy chains may be expressed in bacteria where they assemble into an Fv fragment. Alternatively, the two chains can be engineered to form an interchain disulfide bond to give a dsFv. A single chain Fv (scFv) is a single polypeptide comprising V_{H} and V_{L} sequence domains linked by an intervening linker sequence, such that when the polypeptide folds the resulting tertiary structure mimics the structure of the antigen binding site. See J. S. Huston et al., Proc. Nat. Acad. Sci. U.S.A. 85:5879-5883 (1988). One skilled in the art will recognize that depending on the particular expression method and/or antibody molecule desired, appropriate processing of the recombinant antibodies may be performed to obtain a desired reconstituted or reassembled antibody. See, e.g., Vallejo and Rinas, Biomed Central., available at world wide web URL microbialcellfactories.com/content/3/1/11.

Single domain antibodies are the smallest functional binding units of antibodies (approximately 13 kD in size), corresponding to the variable regions of either the heavy V_{H} or V_{L} chains. See U.S. Pat. No. 6,696,245, WO04/058821, WO04/003019, and WO03/002609. Single domain antibodies are well expressed in bacteria, yeast, and other lower eukaryotic expression systems. Domain deleted antibodies have a domain, such as CH2, deleted relative to the full length antibody. In many cases such domain deleted antibodies, particularly CH2 deleted antibodies, offer improved clearance relative to their full length counterparts. Diabodies are formed by the association of a first fusion protein comprising two V_{H} domains with a second fusion protein comprising two V_{L} domains. Diabodies, like full length antibodies, are bivalent and may be bispecific. Minibodies are fusion proteins comprising a V_{H}, V_{L}, or scFv linked to CH3, either directly or via an intervening IgG hinge. See T. Olafsen et al., Protein Eng. Des. Sel. 17:315-323 (2004). Minibodies, like domain deleted antibodies, are engineered to preserve the binding specificity of full-length antibodies but with improved clearance due to their smaller molecular weight.

The T cell receptor (TcR) is a disulfide linked heterodimer composed of two chains. The two chains are generally disulfide-bonded just outside the T cell plasma membrane in a short extended stretch of amino acids resembling the antibody hinge region. Each TcR chain is composed of one antibody-like variable domain and one constant domain. The full TcR has a molecular mass of about 95 kD, with the individual chains varying in size from 35 to 47 kD. Also encompassed within the meaning of TcR are portions of the receptor, such as, for example, the variable region, which can be produced as a soluble protein using methods well known in the art. For example, U.S. Pat. No. 6,080,840 and A. E. Slanetz and A. L. Bothwell, Eur. J. Immunol. 21:179-183 (1991) describe a soluble T cell receptor prepared by splicing the extracellular domains of a TcR to the glycosyl phosphatidylinositol (GPI) membrane anchor sequences of Thy-1. The molecule is expressed in the absence of CD3 on the cell surface, and can be cleaved from the membrane by treatment with phosphatidylinositol specific phospholipase C (PI-PLC). The soluble TcR also may be prepared by coupling the TcR variable domains to an antibody heavy chain CH2 or CH3 domain, essentially as described in U.S. Pat. No. 5,216,132 and G. S. Basi et al., J. Immunol. Methods 155:175-191 (1992), or as soluble TcR single chains, as described by E. V. Shusta et al., Nat. Biotechnol. 18:754-759 (2000) or P. D. Holler et al., Proc. Natl. Acad. Sci. U.S.A. 97:5387-5392 (2000). Certain embodiments of the invention use TcR “antibodies” as a soluble antibody. The combining site of the TcR can be identified by reference to CDR regions and other framework residues.

The combining site refers to the part of an antibody molecule that participates in antigen binding. The antigen binding site is formed by amino acid residues of the N-terminal variable (V) regions of the heavy (H) and light (L) chains. The antibody variable regions comprise three highly divergent stretches referred to as hypervariable regions or complementarity determining regions (CDRs), which are interposed between more conserved flanking stretches known as framework regions (FRs). The term “region” can refer to a part or portion of an antibody chain or antibody chain domain (e.g., a part or portion of a heavy or light chain or a part or portion of a constant or variable domain), as well as more discrete parts or portions of said chains or domains. For example, light and heavy chains or light and heavy chain variable domains include CDRs interspersed among FRs. The term complementarity determining region (CDR), as used herein, refers to amino acid sequences which together define the binding affinity and specificity of the natural Fv region of a native immunoglobulin binding site. The term framework region (FR), as used herein, refers to amino acid sequences interposed between CDRs. These portions of the antibody serve to hold the CDRs in appropriate orientation (allows for CDRs to bind antigen). The three hypervariable regions of a light chain (LCDR1, LCDR2, and LCDR3) and the three hypervariable regions of a heavy chain (HCDR1, HCDR2, and HCDR3) are disposed relative to each other in three dimensional space to form an antigen binding surface or pocket. In heavy-chain antibodies or V_{H} domains, the antigen binding site is formed by the three hypervariable regions of the heavy chains. In V_{L} domains, the antigen binding site is formed by the three hypervariable regions of the light chain.

The identity of the amino acid residues in a particular antibody that make up a combining site can be determined using methods well known in the art. For example, antibody CDRs may be identified as the hypervariable regions originally defined by Kabat et al. See E. A. Kabat et al., Sequences of Proteins of Immunological Interest, 5.sup.th ed., Public Health Service, NIH, Washington D.C. (1992). The positions of the CDRs may also be identified as the structural loop structures originally described by Chothia and others. See, e.g., C. Chothia and A. M. Lesk, J. Mol. Biol. 196:901-917 (1987); C. Chothia et al., Nature 342:877-883 (1989); and A. Tramontano et al., J. Mol. Biol. 215:175-182 (1990). Other methods include the “AbM definition,” which is a compromise between Kabat and Chothia and is derived using Oxford Molecular's AbM antibody modeling software (now Accelrys), or the “contact definition” of CDRs set forth in R. M. MacCallum et al., J. Mol. Biol. 262:732-745 (1996). Table 2 identifies CDRs based upon various known definitions:

TABLE 2
CDR Definitions

| CDR | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 | H31-H35B | H26-H35B | H26-H32 . . . | H30-H35B |
| (Kabat) | | | H34 | |
| H1 | H31-H35 | H26-H35 | H26-H32 | H30-H35 |
| (Chothia) | | | | |
| H2 | H50-H56 | H50-H58 | H52-H56 | H47-H58 |
| H3 | H95-H102 | H95-H102 | H95-H102 | H93-H101 |

General guidelines by which one may identify the CDRs in an antibody from sequence alone are as follows:

LCDR1:
Start—Approximately residue 24.
Residue before is always a Cys.
Residue after is always a Trp, typically followed by Tyr-Gln, but also followed by Leu-Gln, Phe-Gln, or Tyr-Leu.
Length is 10 to 17 residues.

LCDR2:
Start—16 residues after the end of L1.
Sequence before is generally Ile-Tyr, but also may be Val-Tyr, Ile-Lys, or Ile-Phe.
Length is generally 7 residues.

LCDR3:
Start—33 residues after end of L2.
Residue before is a Cys.
Sequence after is Phe-Gly-X-Gly.
Length is 7 to 11 residues.

HCDR1:
Start—approximately residue 26, four residues after a Cys under Chothia/AbM definitions; start is 5 residues later under Kabat definition.
Sequence before is Cys-X-X-X.
Residue after is a Trp, typically followed by Val, but also followed by Ile or Ala.
Length is 10 to 12 residues under AbM definition; Chothia definition excludes the last 4 residues.

HCDR2:
Start—15 residues after the end of Kabat/AbM definition of CDR-H1.
Sequence before is typically Leu-Glu-Trp-Ile-Gly, but a number of variations are possible.
Sequence after is Lys/Arg-Leu/Ile/IVal/Phe/Thr/Ala-Thr/Ser/Ile/Ala.
Length is 16 to 19 residues under Kabat definition; AbM definition excludes the last 7 residues.

HCDR3:
Start—33 residues after end of CDR-H2 (two residues after a Cys).
Sequence before is Cys-X-X (typically Cys-Ala-Arg).
Sequence after is Trp-Gly-X-Gly.
Length is 3 to 25 residues.

The identity of the amino acid residues in a particular antibody that are outside the CDRs, but nonetheless make up part of the combining site by having a side chain that is part of the lining of the combining site (i.e., that is available to linkage through the combining site), can be determined using methods well known in the art, such as molecular modeling and X-ray crystallography. See, e.g., L. Riechmann et al., Nature 332:323-327 (1988).

Antibodies suitable for use herein may be obtained by conventional immunization, reactive immunization in vivo, or by reactive selection in vitro, such as with phage display. Antibodies may also be obtained by hybridoma or cell fusion methods or in vitro host cells expression system. Antibodies may be produced in humans or in other animal species. Antibodies from one species of animal may be modified to reflect another species of animal. For example, human chimeric antibodies are those in which at least one region of the antibody is from a human immunoglobulin. A human chimeric antibody is typically understood to have variable region amino acid sequences homologous to a non-human animal, e.g., a rodent, with the constant region having amino acid sequence homologous to a human immunoglobulin In contrast, a humanized antibody uses CDR sequences from a non-human antibody with most or all of the variable framework region sequence and all the constant region sequence from a human immunoglobulin. Chimeric and humanized antibodies may be prepared by methods well known in the art including CDR grafting approaches (see, e.g., N. Hardman et al., Int. J. Cancer 44:424-433 (1989); C. Queen et al., Proc. Natl. Acad. Sci. U.S.A. 86:10029-10033 (1989)), chain shuffling strategies (see, e.g., Rader et al., Proc. Natl. Acad. Sci. U.S.A. 95:8910-8915 (1998), genetic engineering molecular modeling strategies (see, e.g., M. A. Roguska et al., Proc. Natl. Acad. Sci. U.S.A. 91:969-973 (1994)), and the like.

The terms “humanized antibody,” as used herein, refers to an antibody that includes at least one humanized immunoglobulin or antibody chain (i.e., at least one humanized light or heavy chain) derived from a non-human parent antibody, typically murine, that retains or substantially retains the antigen-binding properties of the parent antibody but which is preferably less immunogenic in humans. The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and CDRs (e.g., at least one CDR) substantially from a nonhuman immunoglobulin or antibody, and further includes constant regions (e.g., at least one constant region or portion thereof, in the case of a light chain, and preferably three constant regions in the case of a heavy chain).

The term constant region (CR) as used herein, refers to the portion of the antibody molecule which confers effector functions. Typically non-human (e.g., murine), constant regions are substituted by human constant regions. The constant regions of the subject chimeric or humanized antibodies are typically derived from human immunoglobulins. The heavy chain constant region can be selected from any of the five isotypes: alpha, delta, epsilon, gamma, or mu. Further, heavy chains of various subclasses (such as the IgG subclasses of heavy chains) are responsible for different effector functions and thus, by choosing the desired heavy chain constant region, antibodies with desired effector function can be produced. Preferred constant regions are gamma 1 (IgG1), gamma 3 (IgG3) and gamma 4 (IgG4). More preferred is an Fc region of the gamma 1 (IgG1) isotype. The light chain constant region can be of the kappa or lambda type, preferably of the kappa type. In one embodiment the light chain constant region is the human kappa constant chain and the heavy constant chain is the human IgG1 constant chain.

An antibody can be humanized by any method, which is capable of replacing at least a portion of a CDR of a human antibody with a CDR derived from a nonhuman antibody. Methods for humanizing non-human antibodies have been described in the art, examples of which may be found in U.S. Pat. Nos. 5,225,539; 5,693,761; 5,821,337; and 5,859,205; U.S. Pat. Pub. Nos. 2006/0205670 and 2006/0261480; Padlan et al., FASEB J. 9:133-9 (1995); Tamura et al., J. Immunol. 164:1432-41 (2000). Preferably, a humanized antibody has one or more amino acid residues introduced into it from a source which is non-human. These non-human amino acid residues are often referred to as “import” residues, which are typically taken from an “import” variable domain. Humanization can be essentially performed following the methods of Winter and colleagues (see, e.g., P. T. Jones et al., Nature 321:522-525 (1986); L. Riechmann et al., Nature 332:323-327 (1988); M. Verhoeyen et al., Science 239:1534-1536 (1988)) by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. In practice, humanized antibodies are typically human antibodies in which some hypervariable region residues and possibly some framework (FR) residues are substituted by residues from analogous sites in rodent antibodies.

The choice of human variable domains, both light and heavy, to be used in making humanized antibodies is very important to reduce antigenicity and human anti-mouse antibody (HAMA) response when the antibody is intended for human therapeutic use. According to the so-called “best-fit” method, the human variable domain utilized for humanization is selected from a library of known domains based on a high degree of homology with the rodent variable region of interest (M. J. Sims et al., J. Immunol., 151:2296-2308 (1993); M. Chothia and A. M. Lesk, J. Mol. Biol. 196:901-917 (1987)). Another method uses a framework region derived from the consensus sequence of all human antibodies of a particular subgroup of light or heavy chains. The same framework may be used for several different humanized antibodies (see, e.g., P. Carter et al., Proc. Natl. Acad. Sci. U.S.A. 89:4285-4289 (1992); L. G. Presta et al., J. Immunol., 151:2623-2632 (1993)).

Humanized antibodies of the invention also can be produced in a host cell transfectoma using, for example, a combination of recombinant DNA techniques and gene transfection methods as is well known in the art (e.g., Morrison, S., Science 229:1202 (1985)).

For example, to express the antibodies, or antibody fragments thereof, DNAs encoding partial or full-length light and heavy chains, can be obtained by standard molecular biology techniques (e.g., PCR amplification, site directed mutagenesis) and can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Alternatively, nonviral regulatory sequences may be used, such as the ubiquitin promoter or β-globin promoter.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like.

As an alternative to humanization, human antibodies can be generated. For example, it is now possible to produce transgenic animals (e.g., mice) that are capable, upon immunization (or reactive immunization in the case of catalytic antibodies) of producing a fall repertoire of human antibodies in the absence of endogenous immunoglobulin production. For example, it has been described that the homozygous deletion of the antibody heavy-chain joining region (J_{H}) gene in chimeric and germ-line immunoglobulin gene array into such germ-line mutant mice will result in the production of human antibodies upon antigen challenge. See, e.g., B. D. Cohen et al, Clin. Cancer Res. 11:2063-2073 (2005); J. L. Teeling et al., Blood 104:1793-1800 (2004); N. Lonberg et al., Nature 368:856-859 (1994); A. Jakobovits et al., Proc. Natl. Acad. Sci. U.S.A. 90:2551-2555 (1993); A. Jakobovits et al., Nature 362:255-258 (1993); M. Bruggemann et al., Year Immunol. 7:33-40 (1993); L. D. Taylor, et al. Nucleic Acids Res. 20:6287-6295 (1992); M. Bruggemann et al., Proc. Natl. Acad. Sci. U.S.A. 86:6709-6713 (1989)); and WO 97/17852.

Alternatively, phage display technology (see, e.g., J. McCafferty et al., Nature 348:552-553 (1990); H. J. de Haard et al., J Biol Chem 274, 18218-18230 (1999); and A. Kanppik et al., J Mol Biol, 296, 57-86 (2000)) can be used to produce human antibodies and antibody fragments in vitro using immunoglobulin variable domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, and is reviewed in, e.g., K. S. Johnson and D. J. Chiswell, Curr. Opin. Struct. Biol. 3:564-571 (1993). Several sources of V-gene segments can be used for phage display. T. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by J. D. Marks et al., J. Mol. Biol. 222:581-597 (1991) or A. D. Griffiths et al., EMBO J. 12:725-734 (1993). See also U.S. Pat. Nos. 5,565,332 and 5,573,905; and L. S. Jespers et al., Biotechnology 12:899-903 (1994). As indicated above, human antibodies may also be generated by in vitro activated B cells. See, e.g., U.S. Pat. Nos. 5,567,610 and 5,229,275; and C. A. K. Borrebaeck et al., Proc. Natl. Acad. Sci. U.S.A. 85:3995-3999 (1988).

Amino acid sequence modification(s) of the antibodies described herein are contemplated. For example, it may be desirable to improve the binding affinity and/or other biological properties of the antibody. Amino acid sequence variants of an antibody are prepared by introducing appropriate nucleotide changes into the antibody nucleic acid, or by peptide synthesis. Such modifications include, for example, deletions from, insertions into, and/or substitutions of residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of an antibody molecule include the fusion to the N- or C-terminus of an anti-antibody to an enzyme or a polypeptide which increases the serum half-life of the antibody.

Another type of variant is an amino acid substitution variant. These variants have at least one amino acid residue in an antibody molecule replaced by a different residue. The sites of greatest interest for substitutional mutagenesis include the hypervariable regions, but FR alterations are also contemplated. Conservative substitutions are shown in Table 3 below under the heading of “preferred substitutions.” If such substitutions result in a change in biological activity, then more substantial changes, denominated “exemplary substitutions” as further described below in reference to amino acid classes, may be introduced and the products screened.

TABLE 3
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Nle | Leu |
| Leu (L) | Nle; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Nle | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: Nle, Met, Ala, Val, Leu, Ile;

(2) neutral hydrophilic: Cys, Ser, Thr;

(3) acidic: Asp, Glu;

(4) basic: Asn, Gln, His, Lys, Arg;

(5) residues that influence chain orientation: Gly, Pro; and

(6) aromatic: Trp, Tyr, Phe.

Non-conservative substitutions will entail exchanging a member of one of these classes for a member of another class.

Any cysteine residue not involved in maintaining the proper conformation of the antibody may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity). In order to identify candidate hypervariable region sites for modification, alanine scanning mutagenesis can be performed to identify hypervariable region residues contributing significantly to antigen binding. Once such variants are generated, the panel of variants is subjected to screening as described herein and antibodies with superior properties in one or more relevant assays may be selected for further development.

Another type of amino acid variant of the antibody alters the original glycosylation pattern of the antibody by deleting one or more carbohydrate moieties found in the antibody and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences Asn-X″-Ser and Asn-X″-Thr, where X″ is any amino acid except proline, are generally the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of or substitution by one or more serine or threonine residues to the sequence of the original antibody (for O-linked glycosylation sites).

It may be desirable to modify an antibody with respect to effector function, for example to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively, an antibody can be engineered which has dual Fc regions and may thereby have enhanced complement lysis and ADCC capabilities. See G. T. Stevenson et al., Anticancer Drug Des. 3:219-230 (1989).

To increase the serum half life of an antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Various techniques have been developed for the production of whole antibodies and antibody fragments. Traditionally, antibody fragments were derived via proteolytic digestion of intact antibodies (see, e.g., K. Morimoto and K. Inouye, J. Biochem. Biophys. Methods 24:107-117 (1992); M. Brennan et al., Science 229:81-83 (1985)). However, these fragments can now be produced directly by recombinant host cells. Fab, Fv, V_{H}, V_{L}, and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (P. Carter et al., Biotechnology 10:163-167 (1992)). According to another approach, F(ab′)₂ fragments can be isolated directly from recombinant host cell culture.

A variety of expression vector/host systems may be utilized to express antibodies. These systems include but are not limited to microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems.

Expression vectors and host cells suitable for expression of recombinant antibodies and humanized antibodies in particular, are well known in the art. The following references are representative of methods and vectors suitable for expression of recombinant immunoglobulins which may be utilized in carrying out the present invention: Weidle et al., Gene, 51:21-29 (1987); Dorai et al., J. Immunol., 13(12):4232-4241 (1987); De Waele et al., Eur. J. Biochem., 176:287-295 (1988); Colcher et al., Cancer Res., 49:1738-1745 (1989); Wood et al., J. Immunol., 145(9):3011-3016 (1990); Bulens et al., Eur. J. Biochem., 195:235-242 (1991); Beldsington et al., Biol. Technology, 10:169 (1992); King et al., Biochem. J., 281:317-323 (1992); Page et al., Biol. Technology, 9:64 (1991); King et al., Biochem. J., 290:723-729 (1993); Chaudhary et al., Nature, 339:394-397 (1989); Jones et al., Nature, 321:522-525 (1986); Morrison and Oi, Adv. Immunol., 44:65-92 (1989); Benhar et al., Proc. Natl. Acad. Sci. USA, 91:12051-12055 (1994); Singer et al., J. Immunol., 150:2844-2857 (1993); Couto et al., Hybridoma, 13(3):215-219 (1994); Queen et al., Proc. Natl. Acad. Sci. USA, 86:10029-10033 (1989); Caron et al., Cancer Res., 52:6761-6767 (1992); Coloura et al, J. Immunol. Meth., 152:89-109 (1992). Moreover, vectors suitable for expression of recombinant antibodies are commercially available. The vector may, for example, be a bare nucleic acid segment, a carrier-associated nucleic acid segment, a nucleoprotein, a plasmid, a virus, a viroid, or a transposable element.

Host cells known to be capable of expressing functional immunoglobulins include, for example: mammalian cells such as Chinese Hamster Ovary (CHO) cells; bacteria such as Escherichia coli; yeast cells such as Saccharomyces cerevisiae; and other host cells. Mammalian cells that are useful in recombinant antibody expression include but are not limited to VERO cells, HeLa cells, CHO cell lines (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), COS cells (such as COS-7), W138, BHK, HepG2, 3T3, RIN, MDCK, A549, PC12, K562 and 293 cells; myeloma cells, such as NS0 and SP2/0 cells as well as hybridoma cell lines. Mammalian cells are preferred for preparation of those antibodies that are typically glycosylated and require proper refolding for activity. Preferred mammalian cells include CHO cells, hybridoma cells, and myeloid cells. Of these, CHO cells are used by many researchers given their ability to effectively express and secrete immunoglobulins. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, and the like.

To facilitate a better understanding of the present invention, the following examples of specific embodiments are given. In no way should the following examples be read to limit or define the entire scope of the invention.

### EXAMPLES

### Example 1

Cell Lines and Culture. The colon cancer cell line HCT116 (ATCC, the American Type Tissue Collection, #CCL-247) were maintained in Dulbecco's modified Eagle Medium supplemented with 10% FBS. All in vitro experiments were conducted at 60-80% confluence.

SiRNA Constructs and In Vitro Delivery. SiRNA was purchased from OligoEngine (Seattle, Wash.). A non-silencing siRNA sequence was shown by BLAST search to not share sequence homology with any known human mRNA (target sequence 5′-AAUUCUCCGAACGUGUCACGU-3′ (SEQ ID NO:1). SiRNA with the target sequence 5′-UAUCGUGCCACCUGAGAGA-3′ (SEQ ID NO:2), designed and shown to target mRNA of the ZNF306 protein, and was used to downregulate ZNF306 in vitro and in vivo. For in vitro delivery, pSUPERIOR.retro.puro (OilgoEngine, #VEC-IND-0010) vector was used to generate ZNF306 siRNA. Target sequence of ZNF306 was determined by the Oligoengine Workstation 2, which is UAUCGUGCCACCUGAGAGA as shown above. In order to clone the target sequence into pSUPERIOR.retro.puro vector, BglII, HindIII, and Hairpin sequences were added with the target sequence, then forward and reverse sequences were synthesized.

The forward and reverse strands of the oligonucleotides that contain the siRNA-expressing sequence that target mRNA of the ZNF306 protein were annealed. The pSUPERIOR.retro.puro vector was linearized with BglII and HindIII, the annealed oligonucleotides were cloned into the vector. pSUPERIOR.retro.puro-ZNF306-siRNA vector was transfected into a packaging cell line and the harvested purified retrovirus was introduced to HCT116 cells. The cells were subsequently selected with puromycin to establish a stable cell line for siRNA expression. Then, RT-PCR was performed to detect ZNF306 expression. A non-silencing siRNA construct (sequence as above) was used as control for ZNF306 targeting experiments.

Liposomal Preparation. SiRNA for in vivo delivery was incorporated into DOPC (1,2-dioleoylsn-glycero-3-phosphatidylcholine; MD Anderson Cancer Center, Houston, Tex.). DOPC and siRNA were mixed in the presence of excess tertiary-butanol at a ratio of 1:10 siRNA:DOPC (weight:weight). Tween-20 was added to the mixture in a ratio of 1:19 Tween-20:siRNA/DOPC. The mixture was vortexed, frozen in an acetone/dry ice bath, and lyophilized. Prior to in vivo administration, this preparation was hydrated with normal 0.9% saline at a concentration of 15 μg/ml, to achieve the desired dose in 150-200 μl per injection.

Western Blot. Western blotting for the FLAG-tagged ZNF306 was accomplished using either anti-Flag M2 antibody (Sigma Chemicals) (1:5000) or a HRP-conjugated anti-mouse IgG (1:10,000), or anti-ZNF306 that we made (1:10,000) and a HRP-conjugated anti-rabbit IgG secondary antibody (1:10,000). Reactive products were visualized by ECL. Cultured cell lysates were prepared by washing cells with PBS followed by incubation in modified RIPA lysis buffer (50 mM Tris, 150 mM NaCl, 1% triton, 0.5% deoxycholate plus 25 μg/ml leupeptin, 10 μg/ml aprotinin, 2 mM EDTA, and 1 mM sodium orthovanadate (Sigma Chemical Co, St. Louis, Mo.)) for 10 min at 4° C. Cells were scraped from plates, centrifuged at 13,000 rpm for 20 min at 4° C. and the supernatant stored at −80° C. To prepare lysate from snap frozen tissue, approximately 30 mm³ cuts of tissue were incubated on ice in RIPA for 3 hrs, mortar and pestle disrupted and homogenized, centrifuged, and the supernatant stored at −80° C. Samples from 3 regions of the tumor were collected and tested individually. Protein concentrations were determined using a BCA Protein Assay Reagent kit (Pierce Biotechnology, Rockford, Ill.), and subjected to 10% SDS-PAGE separation. Samples transferred to a nitrocellulose membrane by semi-dry electrophoresis (Bio-Rad Laboratories, Hercules, Calif.) were incubated with the appropriate antibody overnight at 4° C., detected with 1 μg/ml HRP-conjugated anti-rabbit IgG (Amersham, Piscataway, N.J.) (if necessary), and developed using enhanced chemiluminescence detection kit (ECL, Pierce). Membranes were tested for β-actin (0.1 μg/ml anti-β-actin primary antibody (Sigma) to confirm equal loading.

Immunohistochemistry. Formalin-fixed, paraffin embedded sections were deparaffinized by sequential washing with xylene, 100% ethanol, 95% ethanol, 80% ethanol, and PBS. Antigen retrieval was performed by heating in steam cooker in 0.2 M tris HCl (pH 9.0) for 20 minutes. After cooling and PBS wash, endogenous peroxide was blocked with 3% H₂O₂ in methanol for 5 mins. Nonspecific proteins were blocked with normal horse and goat serum at 1-5% overnight at 4° C. Slides were incubated in primary antibody (1:10,000 of rabbit anti-ZNF306 antiserum) for 4 hrs at 4° C., washed, followed incubation with a HRP-coupled anti-rabbit antibody for 1 hr at room temperature. Immunoreactivity was detected with DAB (Phoenix Biotechnologies, Huntsville, Ala.) substrate for 7 minutes, and counterstained with Gil No.3 hematoxylin (Sigma) for 20 secs.

Statistical Considerations. For in vivo therapy experiments, 10 mice in each group were used, as directed by a power analysis to detect a 50% reduction in tumor size (beta error 0.8). Mean tumor size was analyzed for statistical significance (achieved if p<0.05) with student's t-test if values were normally distributed, otherwise with the Mann-Whitney rank sum test, using STATA 8 software (College Station, Tex.).

Anoikis Assays. Cells (5×10⁴/well) were cultured in 6-well ultra-low cluster (ULC) plates (Costar, #3471) with a covalently bound hydrogel layer (Polystyrene) that effectively inhibits cell attachment. After 2 days, cells were collected and subjected to FACS analysis as described by us elsewhere (Yan, C., Lu, D., Hai, T., Boyd, D. D. (2005). ATF3, a stress sensor, activates p53 by blocking its ubiquitination. European Molecular Biology Organization 24, 2425-2435). Apoptotic cells were defined as the sub-G1 cell population. Briefly, cells were fixed with cold 70% ethanol and washed once with PBS. The cells were incubated with propidium iodide 50 μg/ml (final concentration) and 20 μg/ml RNAse A (final concentration) at 37° C. for 20 min prior to FACS analysis.

CAST-ing. Flag-ZNF306 protein was purified from HCT116 cells stably expressing the exogenous ZNF306 coding sequence. Briefly, cell lysates were prepared from 90% confluent cells using a lysis buffer (50 mM Tris HCl, pH 7.4, 150 mM NaCl, 1 mM EDTA, and 1% Triton X-100). After thoroughly suspending the anti-Flag M2 affinity gel, 40 μl were transferred and washed 2× with TBS. Cleared clear cell lysate (1 ml) was added to the washed resin and gently shaken at 4° C. overnight. Bound Flag-ZNF306 was then eluted using a 3× tandem repeated Flag peptide.

A random oligonucleotide library (complexity=4×10¹⁵) was synthesized as 5′-CACGTGAGTTCAGCGGATCCTGTCGNNNNNNNNNNNNNNNNNNNNNNNNNGAGGCGAATTCAGTGCAACTGCAGC-3′. (SEQ ID NO:3) Binding reactions contained 2 μl of 10× binding buffer, 2 μl poly dI.dC (2 μg), 10 μl Flag-ZNF306-resin, 10 μg acetylated BSA and 500 ng random oligonucleotides and complexes formed at room temperature for 20 min. After washing with the binding buffer, the following components were added: 300 μl TE buffer, 15 μl of 10% SDS, 7.5 μl of proteinase K (10 mg/ml). After an overnight incubation at 37° C., DNA was extracted 2× with phenol/chloroform, precipitated with ethanol/sodium acetate and finally dissolved in 30 μl TE buffer. PCR was then used to enrich the bound-DNA using a reaction system containing 5 μl DNA, 4 μl dNTP (2 mM), 100 ng each of primers, 10× PCR buffer (5 μl), Taq enzyme (1 μl), and 33 μl H₂O. Amplification was carried out for 15 cycles (94° C., 1 min 62° C., 1 min; and 72° C., 1 min). The 76′-mer PCR product was purified using the Qiagen DNA extraction kit.

The purified DNA was then subjected to 5 more rounds of binding and amplification as described above. In the final round of amplification, DNA was labeled with dCTP-32P and subjected to EMSA using 100 ng purified Flag-ZNF306 protein. The gel was subjected to autoradiography, oligonucleotides in DNA-protein complexes recovered, cloned into the pGEM-T Easy Vector (Promega, #A1360), and finally sequenced using the T7 primer.

Chromatin Immunoprecipitation Assays. Binding of ZNF306 to endogenous target genes was determined using these assays as described by us previously (Yan, C., Wang, H., Toh, Y., Boyd, D. D. (2003). Repression of 92-kDa type IV collagenase expression by MTA1 is mediated through direct interactions with the promoter via a mechanism, which is both dependent on and in-dependent of histone deacetylation. Journal of Biology Chemistry 278, 2309-2316; Wang, H., Yang, L., Jamaluddin, M. d. S., Boyd, D. D. (2004). The Kruppel-like KLF4 transcription factor, a novel regulator of urokinase receptor expression, drives synthesis of this binding site in colonic crypt luminal surface epithelial cells. Journal of Biology Chemistry 279, 22674-22683). Primers within 100 base pairs of the putative ZNF306 binding site were employed. The amount of immunoprecipitated promoter was quantified by real-time PCR as has been previously published (Yan, C., Wang, H., Toh, Y., Boyd, D. D. (2003). Repression of 92-kDa type IV collagenase expression by MTA1 is mediated through direct interactions with the promoter via a mechanism, which is both dependent on and in-dependent of histone deacetylation. Journal of Biology Chemistry 278, 2309-2316).

EMSA. Mobility shift assays were performed as described by us elsewhere (Wang et al., 2004) using 10 μg nuclear extract, 0.6 μg of poly dI/dC and (2×10⁴ cpm) of a [γ³²P] ATP T4 polynucleotide kinase-labeled oligonucleotide.

Growth in Semi-Solid Media. Equal volumes of 1% melted agar (DNA grade) and 2× DMEM/F12 were mixed (40° C.) and 1.5 ml dispensed into a 35 mm dish. A 0.7% Agar solution was maintained at 40° C. is mixed with a warmed 2× DMEM/F12 solution supplemented with 8×10⁴ cells and 1.5 ml dispensed onto the 0.5% agar in the 35 mm dishes. The cultures were incubated at 37° C. in humidified incubator for 10-14 days.

Northern Blotting. Northern blotting was carried out as described by us Wang et al., 2004 using a random primed cDNA specific for the ZNF306 transcript or cDNAs specific for the genes identified in the expression profiling experiments. Stringencies were performed at 65° C. using 0.1×SSC/0.1% SDS.

Orthotopic Tumor Growth. These experiments were carried out as described by Morikawa et al., 1988. (Morikawa, K., Walker, S., Jessup, J., Fidler, I. (1988). In vivo selection of highly metastatic cells from surgical specimens of different primary human colon carcinomas implanted into nude mice. Cancer Research 48, 1943-1948.) Male athymic nude mice (NCI-nu/nu) were maintained under specific pathogen-free (SPF) conditions in facilities approved by the American Association for Accreditation of Laboratory Animal. Cells were harvested with trypsin from sub-confluent cultures, washed once with serum-free medium and resuspended in HBSS. Only single cell suspensions showing a >90% viability will be used. Then, 10⁶ cells in 50 μL of HBSS were injected into the cecal wall of the nude mice (8-12 weeks old) as described by Morikawa et al., 1988. After varying times, mice were sacrificed, tumors harvested and weighed and analyzed by RT-PCR for ZNF306 expression.

Quantitative PCR. Real-time RT-PCR to measure ZNF306 mRNA levels were performed as described by Yan et al., 2003 (Yan, C., Wang, H., Toh, Y., Boyd, D. D. (2003). Repression of 92-kDa type IV collagenase expression by MTA1 is mediated through direct interactions with the promoter via a mechanism, which is both dependent on and in-dependent of histone deacetylation. Journal of Biology Chemistry 278, 2309-2316.) and Yan et al., 2004 (Yan, C., Wang, H., Aggarwal, B. B., Boyd, D. D. (2004). A novel homologous recombination system to study 92 kDa type IV collagenase transcription demonstrates that the NFkB motif drives the transition from a repressed to an activated state of gene expression. FASEB Journal 18, 540-541.) We used primers to detect either the endogenous transcript (5′-GGC CCT GAC CCT CAC CCC-3′ (SEQ ID NO:4) and 5′-CAG ATG TGC CGC CTC CCT CC-3′ (SEQ ID NO:5) located in exons 5 and 6) or the exogenous FLAG-tagged ZNF306 mRNA (5′GACGATGACGACAAGGGATCC 3′ (SEQ ID NO:6) and 5′ CCAGCAGCTCCAGGATCTGC 3′ (SEQ ID NO:7) with the former primer complementary to the FLAG tag). Two controls were used to confirm specificity of the amplification: the presence of a (a) 295 base pair amplified product as determined in gel electrophoresis (b) single peak in melting curves conducted after PCR amplification.

Retroviral Transductions. 100-mm plates containing AmphoPack™-293 cells (Clontech, #631505) were transfected with 10 μg of pLIB-neo-Flag-ZNF306 using Lipofectamine 2000. After 10-24 h, medium was aspirated, the cells washed 2× with PBS, and replenished with 5 ml of fresh cultured medium. Culture supernatant (containing the Flag-ZNF306-encoding retrovirus) was collected every 12 h thereafter for a 48 h period, filtered through a 0.45 μm filter and diluted 2 fold with fresh medium. Actively dividing target cells were then transduced with the filtered virus-containing conditioned medium with the addition of polybrene (final concentration=4 μg/ml).

Transfections. Cells were transfected with 12 μg pIRES2-EGFP-Flag-ZNF306, or the empty vector using Lipofectamine according to the manufacturer's (Invitrogen) instructions. Briefly, 12 μg DNA was diluted into 750 μl Opti-MEM I Reduced Serum Medium without serum. 30 μl Lipofectamine 2000 was also diluted into 750 μl Opti-MEM I Reduced Serum Medium without serum and incubating at room temperature for 5 minutes. Then, the diluted DNA was mixed with the Lipofectamine at room temperature for 20 minutes. The 1.5 ml of DNA-lipofectamine complex was then added to ˜107 cells. After 48 h, cells were selected with 2 mg/ml G418.

For transient transfections to identify optimal siRNA sequences in HCT116 cells, the procedure described by Invitrogen was used (http://www.invitrogen.com/downloads/HCT116_stealthrnai_tsf_protocol.pdf). Transfection efficiency was determined by tranfecting the empty pIRES2-EGFP vector and determining the % of fluorescent cells.

Incorporation of siRNA into liposomes. An efficient delivery vehicle is necessary for in vivo delivery. Cationic liposomes, while efficiently taking up nucleic acids, have had limited success for in vivo gene downregulation, perhaps because of their stable intracellular nature and resultant failure to release siRNA contents. DOPC was selected because another group has successfully used this molecule to deliver antisense oligonucleotides in vivo (Gutierrez-Puente, 1999).

### Example 2

### Elevated ZNF306 mRNA Levels in Colon Cancer

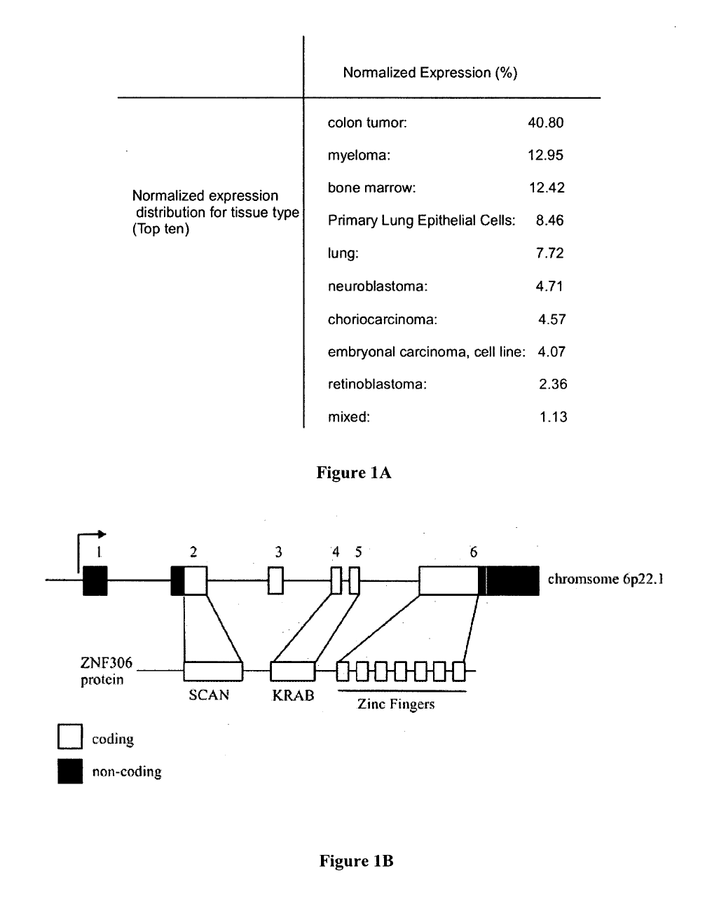

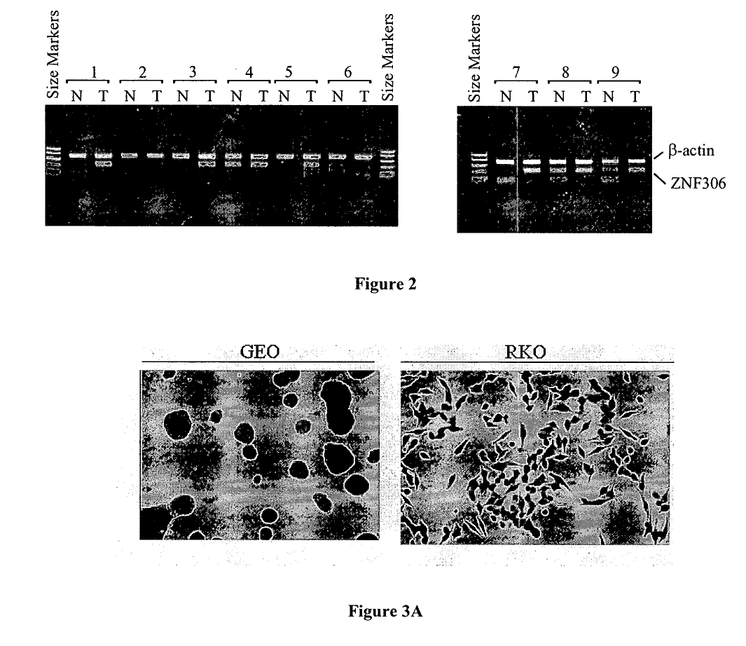

Data-mining of the Unigene Cluster expression database (2004 release) had indicated that the normalized expression of ZNF306 mRNA was highest in colon cancer relative to a composite set of tissues including both normal and other malignant tissues (FIG. 1A). To confirm these observations, total RNA was extracted from 9 colorectal cancers and adjacent non-malignant mucosa. ZNF306 levels were semi-quantitated by RT-PCR using primers specific for this transcript (FIG. 2). An amplified band of the predicted size (294 bp) was detected in 8 of the 9 tumor (T) tissues. Interestingly, of the 9 sets of tissues, ZNF306 mRNA amounts were elevated in 8 of the 9 cancers when compared with the paired non-malignant mucosa (#1, 2, 3, 4, 5, 7, 8, 9). For the remaining patient (#6), ZNF306 mRNA was decreased in the tumor tissue. It appears from the data-mining observations that ZNF306 mRNA levels are indeed elevated in colorectal cancers.

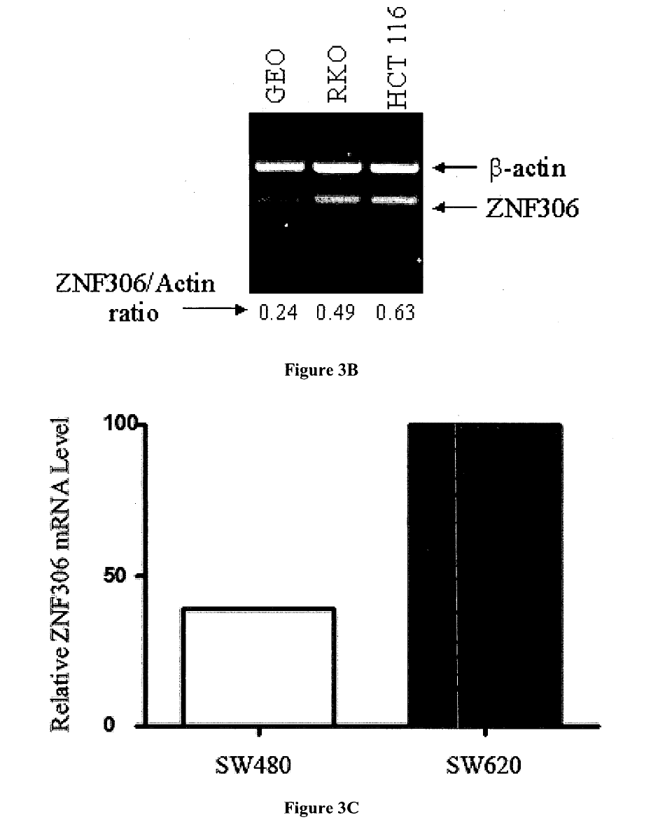

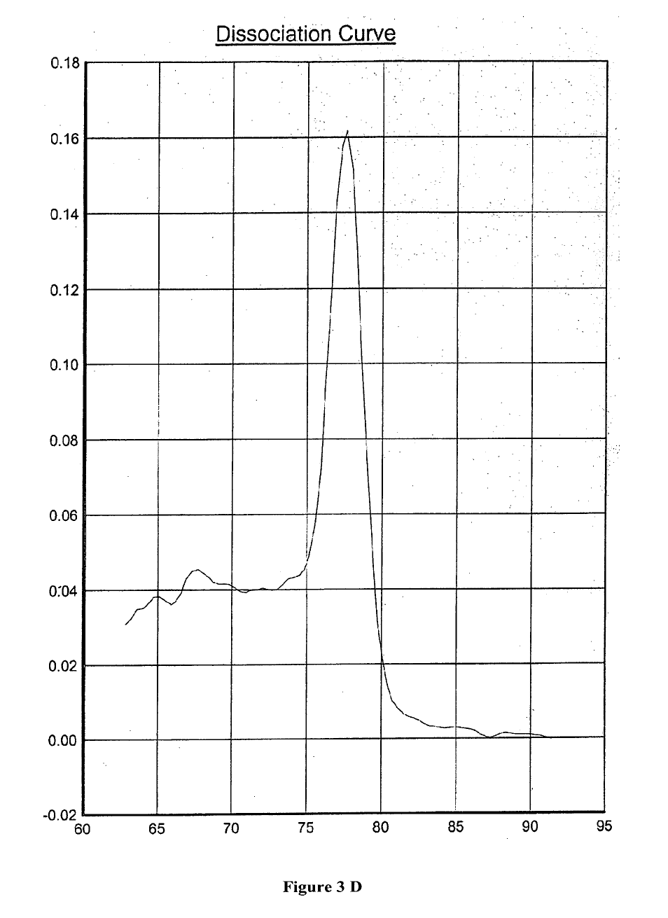

ZNF306 mRNA levels were then measured in cultured colon cancer cells. To address the issue of whether ZNF306 mRNA amounts increase in the progression from well differentiated to poor differentiation, ZNF306 transcript in 3 colon cancer cell lines with varied differentiation status was quantified (Brattain, M. G., Levine, A., Chakrabarty, S., Yeoman, L., Willson, J., Long, B. (1984). Heterogeneity of human colon carcinoma. Cancer Metastasis Reviews 3, 177-191; Chantret, I., Barbat, A., Dussaulx, E., Brattain, M. G., Zweibaum, A. (1988). Epithelial polarity, villin expression, and enterocytic differentiation of cultured colon carcinoma cells: A survey of twenty cell lines. Cancer Research 48, 1936-1942). GEO colon cancer cells (FIG. 3A) are well differentiated as evidenced by their tight junctions and a polarized monolayer with an apical brush border (Chantret et al., 1988). Additionally, these cells can undergo enterocytic differentiation (Chantret et al., 1988). In contrast, the HCT116 and RKO colon cancer cell lines (FIG. 3A) are poorly differentiated (Brattain et al., 1984) and demonstrate high tumorigenecity in vivo (Brattain et al., 1981). RT-PCR semi-quantitation of ZNF306 mRNA levels (FIG. 3B) revealed the lowest level of this transcript in the well differentiated GEO cells with a ZNF306/actin ratio of 0.24 when compared with 0.49 and 0.63 for the poorly differentiated RKO and HCT116 cells respectively. Secondly, to address the issue of whether ZNF306 mRNA levels were elevated in colon cancer cells derived from a metastatic site compared with tumor cells derived from the primary site, ZNF306 mRNA levels in SW480 and SW620 colon cancer cells established from the same patient were compared, with the former derived from the primary tumor and the latter representing tumor cells cultured from a lymph node metastases. Using real-time PCR (FIG. 3C, 3D), the SW620 cells derived from the secondary site showed about a 2.5 fold increase in ZNF306 mRNA amounts compared with the SW480 cells originally generated from the primary tumor. A melting curve of the amplified products (FIG. 3D) revealed a single peak indicative of the specificity in the amplification. Together, these findings indicate that ZNF306 mRNA levels are elevated in concordance with tumor progression.

### Example 3

### Exogenous Expression of ZNF306 Increases Anchorage-Independent Growth

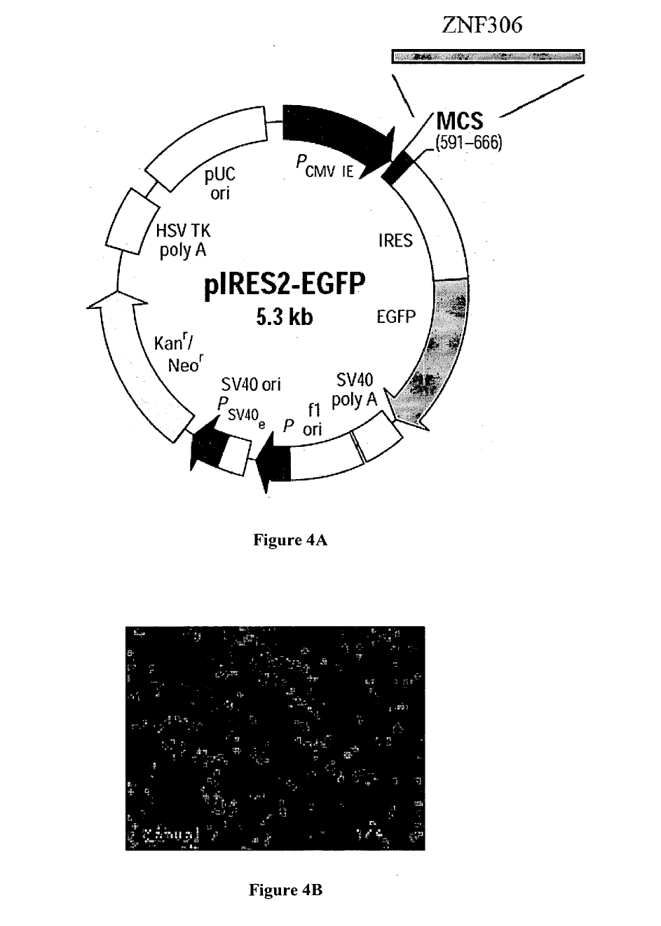

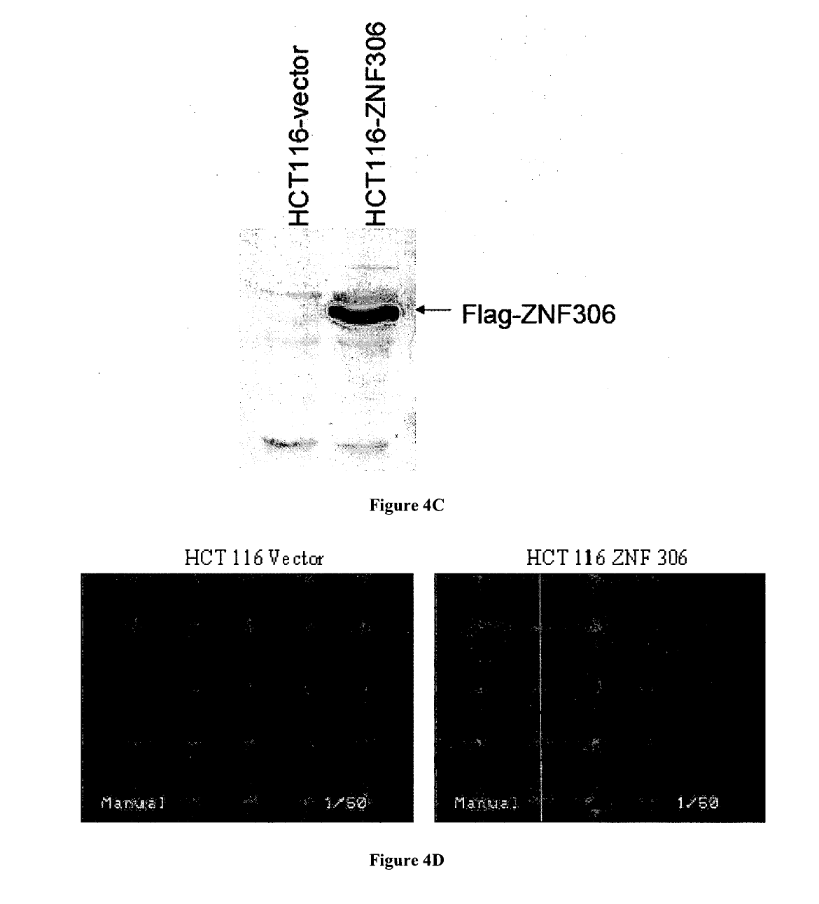

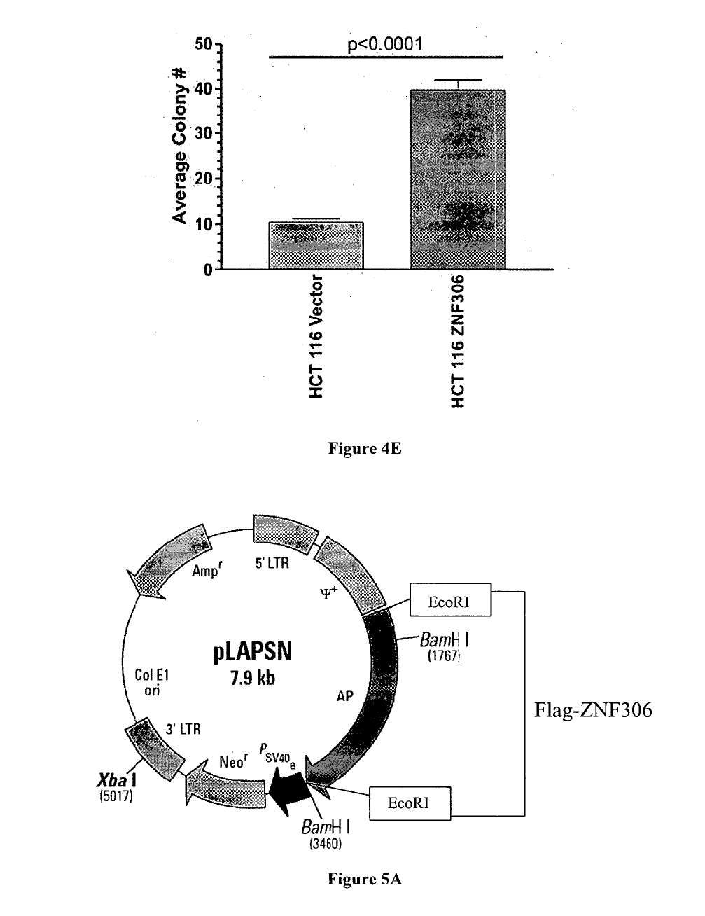

The elevated ZNF306 mRNA levels in the resected colorectal cancers and the progressed cultured colon cancer could either be causal for tumorigenecity/progression or simply represent a consequence. To determine if ZNF306 plays a contributory role in the biology of colon cancer, the full length flag-tagged ZNF306 coding sequence was first cloned from a colon expression library and then subcloned (FIG. 4A) into a bicistronic expression vector (pIRES2-EGFP) which allows for the translation of the EGFP and ZNF306 coding sequences from the same transcript. HCT116 colon cancer cells were transfected with this construct or the empty vector, and G418-resistant clones expanded. Fluorescence microsocopy (FIG. 4B) and Western blotting (FIG. 4C) using an anti-Flag antibody confirmed the successful expression of the exogenous ZNF306 in HCT116 cells.

Anchorage-independent growth is a hallmark of tumorigenicity so we then determined the effect of the exogenously expressed ZNF306 on this parameter. Expectedly, HCT116 cells harboring the empty bicistronic vector generated colonies in semi-solid medium albeit modestly (approximately 10 colonies per field—FIGS. 4D, 4E). However, in contrast, a HCT116 clone expressing the exogenous ZNF306 cDNA showed robust activity in this assay manifesting about a 4 fold increase in colony number. Moreover, the size of the colonies was substantially larger than the vector control (FIG. 4D).

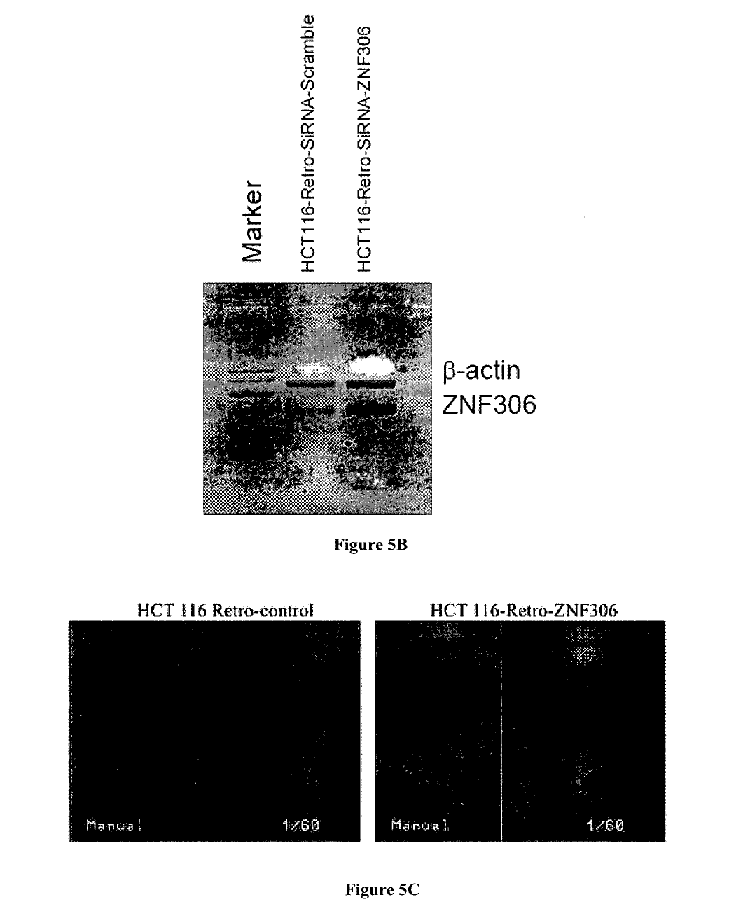

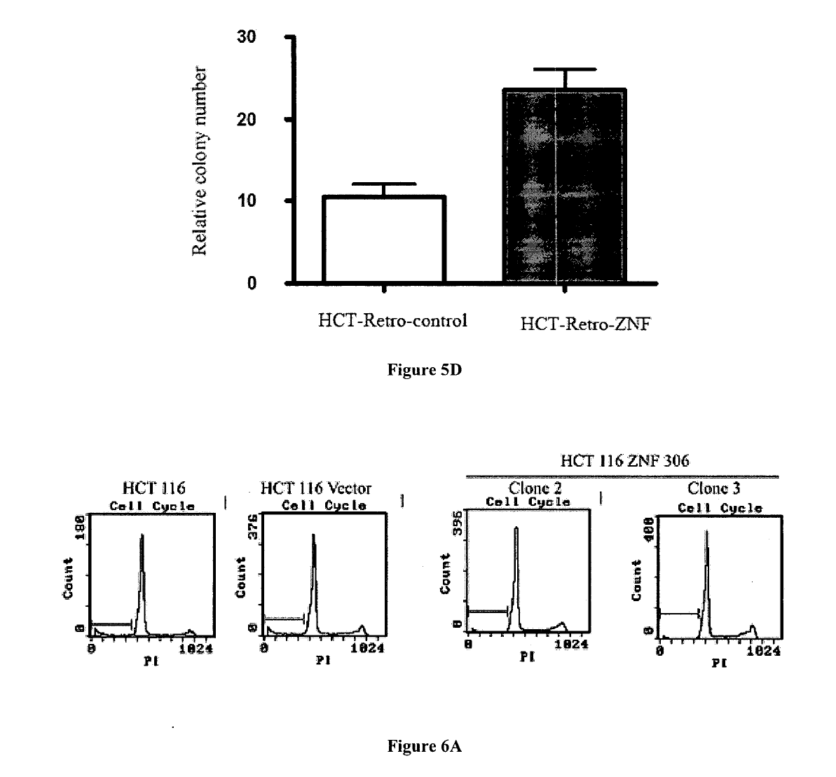

To rule out the possibility that the data reflected clonal variation rather than being a consequence of the expression of the exogenous construct, the flag-tagged ZNF306 was subcloned into the pLAPSN vector (FIG. 5A) and following transfection of 293 cells, the viral supernatant used to transduce the HCT116 cells. Expression of the ZNF306 in the transduced HCT116 colon cancer cells was confirmed by RT-PCR blotting (FIG. 5B). More importantly, and similar to the previous experimental data, a robust stimulation of growth in suspension cultures was seen in the HCT116 cells made to express the exogenous ZNF306 by viral transduction (FIGS. 5C, 5D). These data suggest that ZNF306 increases the in vitro tumorigenecity of the HCT116 colon cancer cells.

### Example 4

### ZNF306 Expression Renders HCT116 Cells Anoikis-Resistant

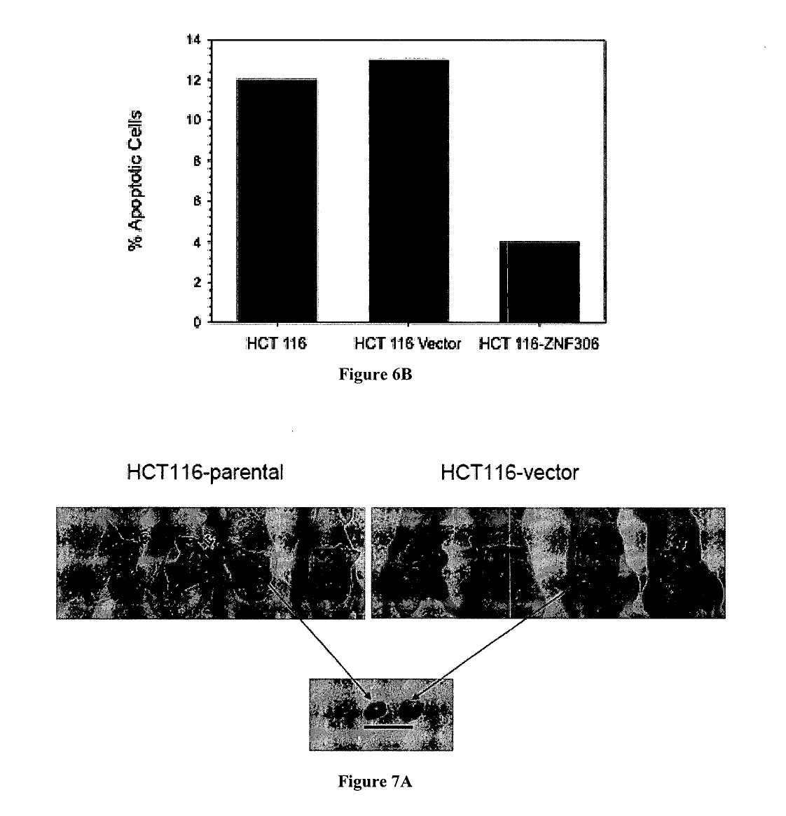

Anoikis (detachment-induced cell death) is a prerequisite for tumor progression since dissemination of malignant cells is dependent on their survival in the vascular and lymphatic systems (Wang, L. H. (2004). Molecular signaling regulating anchorage-independent growth of cancer cells. Mount Sanai Journal of Medicine 71, 361-367; Valentijn, A. J., Zouq, N., Gilmore, A. P. (2004). Anoikis. Biochemical Society Transactions 32 (Pt3), 421-425). Accordingly, we next determined if ZNF306 expression rendered cells resistant to this phenomenon. HCT116 cells overexpressing the exogenous ZNF306 or the vector were sub-cultured on hydrogel-coated plates thereby hindering cell attachment. Quantitation of apoptotic cells showed an apoptotic rate of approximately 12% for the parental HCT116 cells and the vector controls while two independent clones expressing the exogenous ZNF306 showed a 3 fold reduction in this parameter (FIGS. 6A, 6B). Thus, one hallmark of malignancy (growth in semi-solid media) and one parameter of tumor progression (resistance to anoikis) are both augmented by ZNF306 over-expression.

### Example 5

### Orthotopic Implantation of ZNF306-Expressing Colon Cancer Cells Yields Large Tumors

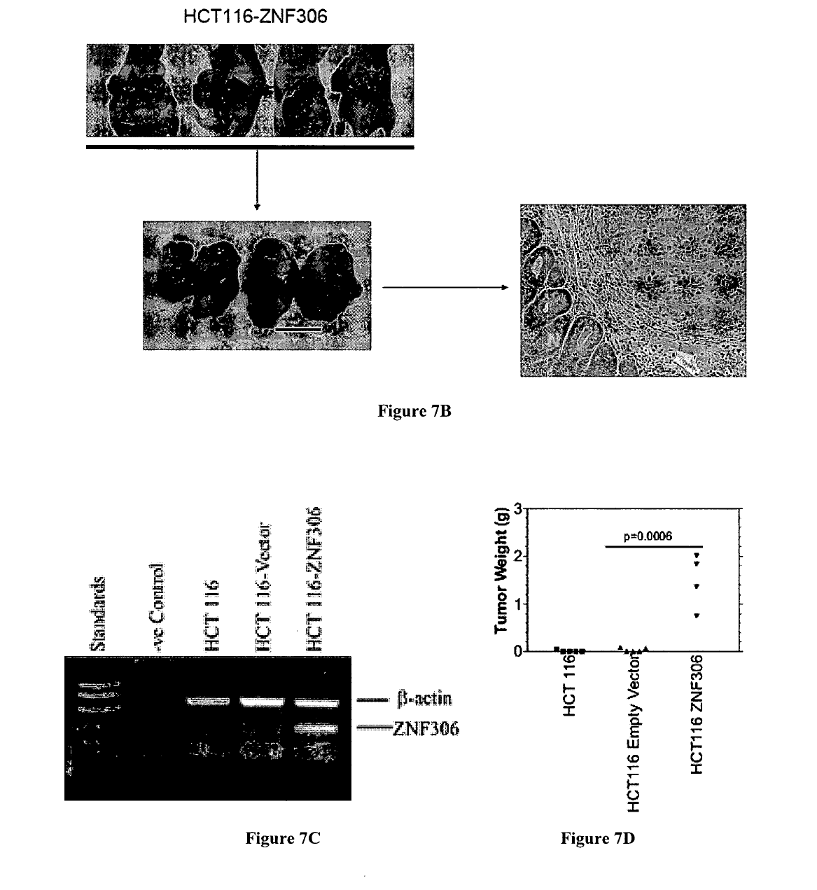

To corroborate these tissue culture observations, tumor growth studies in vivo were performed. Nude mice were injected intracecally with 10⁶ parental HCT116 cells or a pool of HCT116 clones stably expressing the empty pIRES2 EGFP vector or the ZNF306 coding sequence. Parental HCT116 or cells harboring the empty vector were weakly tumorigenic (FIG. 7A) with only 2 of the 8 mice giving rise to tumors in the colon in 7 weeks. In contrast, of the 5 mice injected orthotopically with an equivalent number of HCT116 cells expressing the exogenous ZNF306, all animals showed histologically-confirmed tumors in the colon (FIG. 7B) which were substantially larger in size than those generated with the parental or vector-bearing HCT116 cells. The difference in tumor size was clearly evident in the very disparate tumor weights (FIG. 7D) between the HCT116 ZNF306 and the parental/vector groups (p=0.0006). RT-PCR confirmed the sustained expression of the ZNF306 cDNA in the tumors derived from the pooled HCT116 transfectants stably over-expressing ZNF306 (FIG. 7C).

### Example 6

### SiRNA-Targeting of the Endogenous ZNF306 Transcript Reduces Growth of HCT116 Cells

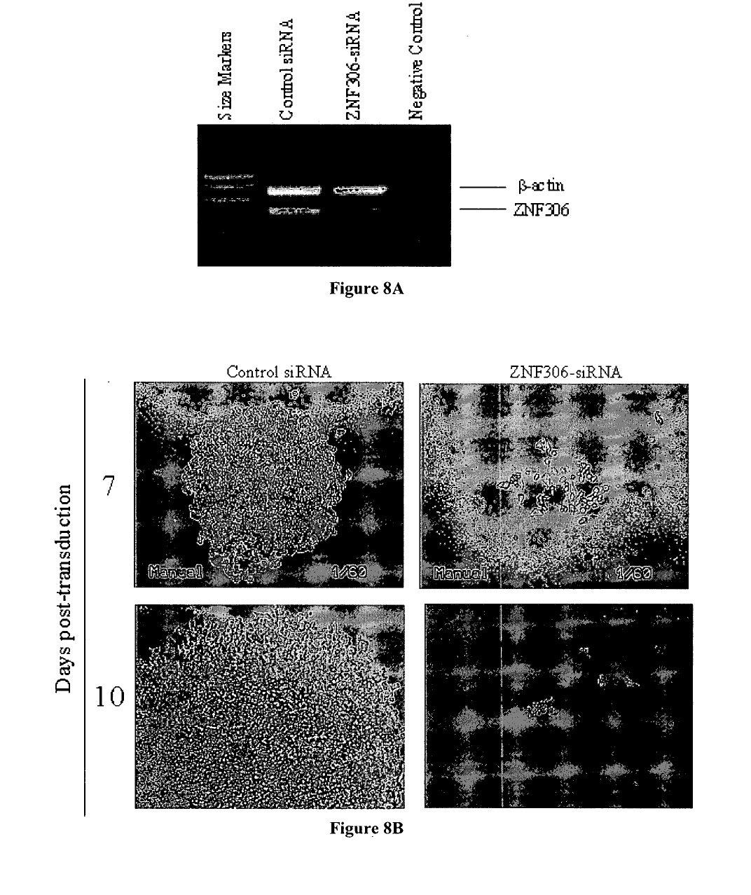

The aforementioned data indicated that increased expression of ZNF306 increases the tumorigenic potential of colon cancer cells. We then determined if interfering with the endogenous ZNF306 transcript would have the opposite effect on the biology. To this end, HCT116 cells were transduced with a virus bearing a siRNA targeting the ZNF306 transcript. The efficacy of this siRNA in reducing endogenous ZNF306 mRNA levels is evident in FIG. 8A. Remarkably, HCT116 cells made to express this anti-ZNF306 siRNA showed drastically reduced colony size (FIG. 8B).

### Example 7

### Subcellular Localization of ZNF306

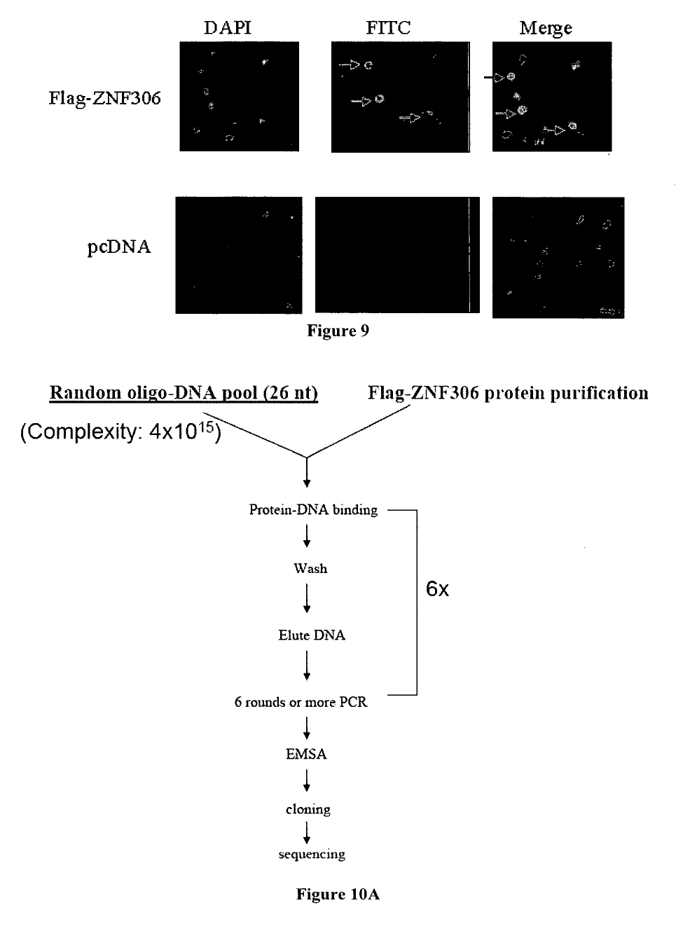

The subcellular localization of the ZNF306 protein was then determined. Although the predicted protein sequence of ZNF306 indicates the presence of several domains usually restricted to transcription factors (zinc fingers, KRAB and SCAN domains), on the other hand, computer analysis did not reveal a nuclear localization signal. Accordingly, HCT116 cells were transiently transfected with the pcDNA3 vector bearing the Flag-tagged ZNF306 coding sequence. Cells were permeabilized and subjected to immunofluorescence studies using an anti-Flag antibody. The expressed protein was readily detected in the nuclei (FIG. 9-arrows) of HCT116 colon cancer cells transiently transfected with the vector bearing the ZNF306 coding sequence but not cells expressing the empty vector (FIG. 9). These data strongly suggest that the ZNF306 is translocated to the nuclear compartment presumably, via a chaperone as described for other transcription factors and histones (Lees and Whitelaw, 1999; Korber and Horz, 2004).

### Example 8

### Cyclic Amplification and Selection of Targets (CAST-ing)

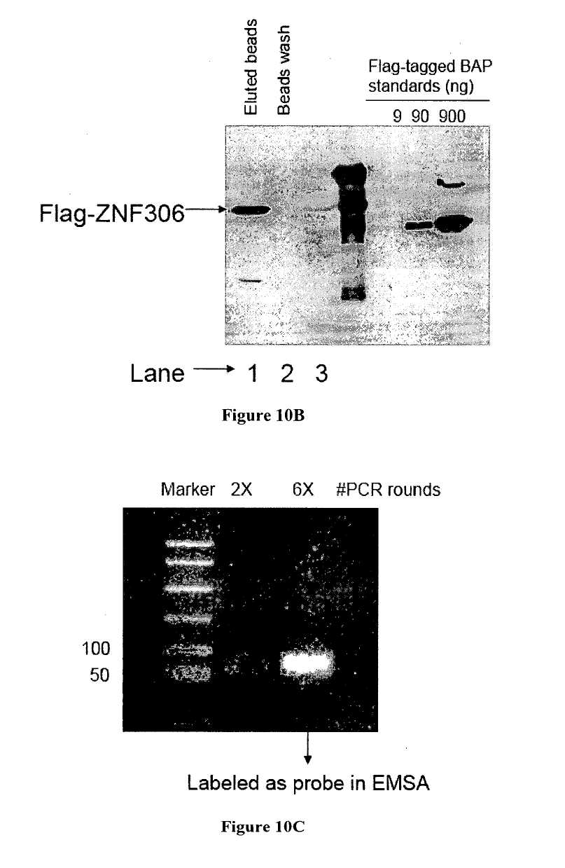

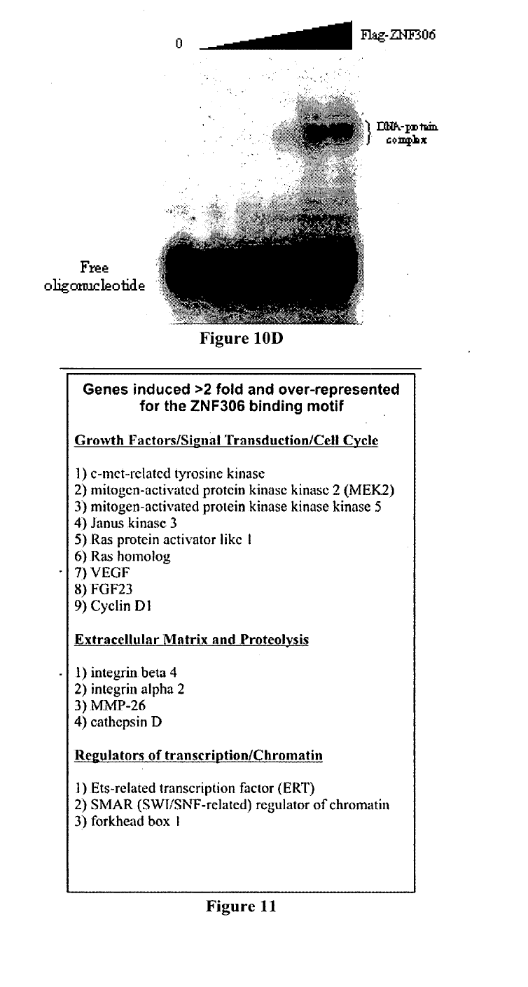

The nuclear localization of ZNF306 together with its structural features strongly argue that this protein is a transcription factor regulating gene expression. However, the DNA recognition motif for ZNF306 is unknown. Consequently Cyclic Amplification and Selection of Targets (CAST-ing) was performed to identify a putative DNA-binding sequence(s). Purified immobilized Flag-tagged ZNF306 protein (FIG. 10B) was incubated with an oligonucleotide library (complexity of 4×10¹⁵) harboring 26-mers of random sequence (FIG. 10A). Protein-DNA complexes were washed, the DNA eluted and amplified by PCR. This procedure was repeated six times (FIGS. 10A, C). In the final round of PCR, ZNF306-binding oligonucleotides were radiolabeled and subjected to EMSA with the ZNF306 protein (FIG. 10D). This data indicate the ability of the ZNF306 protein to bind DNA.

### Example 9

### Expression Profiling to Identify Genes Whose Expression are Altered by ZNF306 Over-Expression

RNA was extracted from orthotopically-established tumors (as described in FIG. 7) derived from HCT116 cells harboring the empty vector or made to express the ZNF306 coding sequence and subjected to expression profiling using the U133A Affymetrix chip harboring ˜18,000 cDNAs. The tumor material was used instead of monolayer cells since the pro-tumorigenic effects of the ZNF306 are so clearly evident in the in vivo model. FIG. 11 lists some of the genes showing more than 2 fold increased expression in the tumors derived from HCT116 cells stably expressing the exogenous ZNF306.

Of particular interest was the induced expression of a diverse set of genes involved in signal transduction and growth control including a c-met-related tyrosine kinase, Janus kinase 3, a Ras activator and a homolog as well as signaling kinases in the MAPK pathways all with well-established roles in driving tumor cell growth (Jeffers, M., Rong, S., Vande Woude, G. F. (1996). Enhanced tumorigenicity and invasion-metastasis by hepatocyte growth factor/scatter factor-Met signalling in human cells concomitant with induction of the urokinase pro-teolysis network. Molecular and Cellular Biology 16, 1115-1125; Jeffers, M., Schmidt, L., Nakaigawa, N., Webb, C. P., Weirich, G., Kishida, T., Zbar, B., Vande Woude, G. F. (1997). Activating mutations for the Met tyrosine kinase receptor in human cancer. Proceedings of the National Academy of Sciences USA 94, 11445-11450; Abounader et al., J. Natl Cancer Instit., 91, 1548-1556, 1999; Al-Rawi, M. A., Rmali, K., Watkins, G., Mansel, R. E., Jiang, W. G. (2004). Aberrant expression of interleukin-7, (IL-7) and its signaling complex in human breast cancer. European Journal of Cancer 40, 494-502.). The induction of VEGF was also noteworthy considering its well-established role in angiogenesis (Bergers, G., Brekken, R., McMahon, G., Vu, T. H., Itoh, T., Tamaki, K., Tanzawa, K., Thorpe, P., Itohara, S., Werb, Z., Hanahan, D. (2000). Matrix metalloproteinase-9 triggers the angiogenic switch during carcinogenesis. Nature Cell Biology 2, 737-744; Lee, S., Jilani, S. M., Nikolova, G. V., Carpizo, D., Iruela-Arispe, M. L. (2005). Processing of VEGF-A by matrix metalloproteinases regulates bioavailability and vascular patterning in tumors. Journal of Cell Biology 169, 681-691.) a prerequisite for tumor growth in vivo. The elevated expression of TCF-3 and cyclin D1 was also particularly compelling considering that the former is one of the transcription factor effectors of the Wnt pathway (implicated in colon carcinogenesis) with the latter representing a downstream target (Malbon, C. C., Wang, H., Moon, R. T. (2001). Wnt signaling and heterotrimeric G-proteins: strange bedfellows or a classic romance? Biochemical and Biophysical Research Communications 287, 589-593; Radtke, F., Clevers, H. (2005). Self-renewal and cancer of the gut: Two sides of a coin. Science 307, 1904-1909.) well known for its contribution to cell cycle progression. Finally, the elevated expression of MMP-26 and cathepsin D, two proteases implicated in tumor growth and progression, was evident in the ZNF306-expressing tumors.

### Example 10

### Effect of Silencing ZNF306 Expression on Colon Tumorigenesis or Tumor Progression In Vitro and In Vivo

Cell lines which express the endogenous ZNF306 (see FIG. 3) were employed to determine the effect of silencing ZNF306 expression on colon tumorigenecity in vitro and in vivo. First, siRNA sequences were designed using the OligoEngine Workstation 2 program (OligoEngine, Seattle Wash.) targeting sequences unique to the ZNF306 transcript. 3 independent ZNF306 siRNAs were tested for their ability to transiently repress ZNF306 mRNA levels as measured by quantitative RT-PCR. Towards this end, the HCT116 cells were employed, using a transfection procedure optimized for delivery of siRNA into these cells.

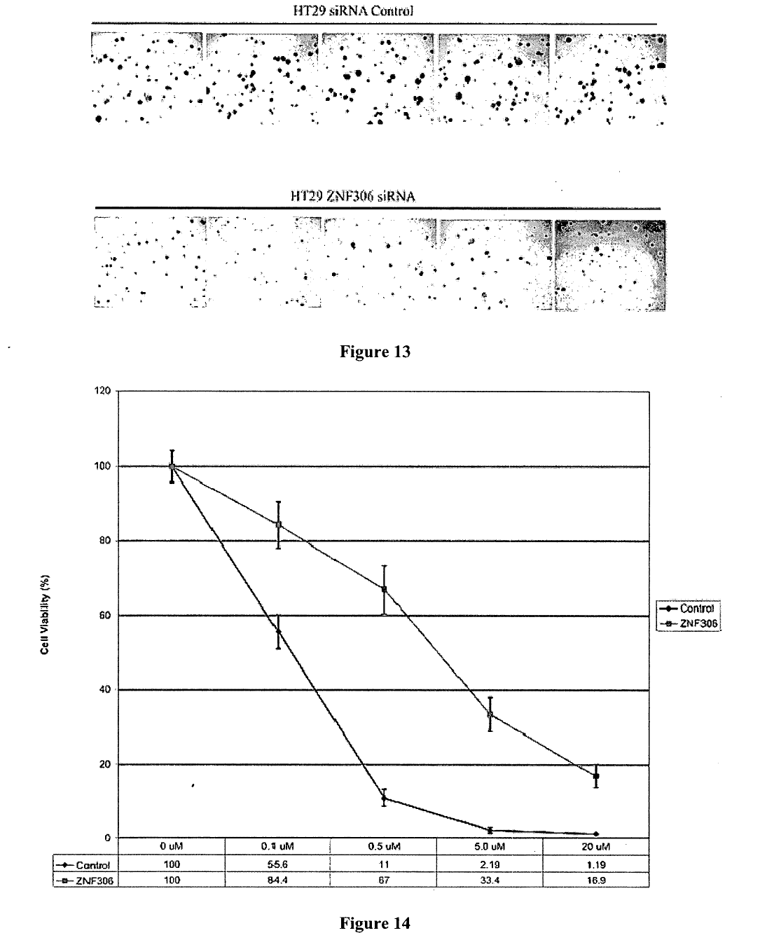

We then subcloned the most effective siRNA, or as a control scrambled siRNA, into the pSUPERIOR retroviral vector (OligoEngine) and transduced the ZNF306-expressing colon cancer cells. RT-PCR was used to confirm that the endogenous ZNF306 mRNA has indeed been knocked down by 75% or greater in the cells transduced with the retrovirus encoding the ZNF306-targeting siRNA compared with retrovirus encoding the scrambled siRNA sequence. Colon cancer cells repressed for ZNF306 expression were then assayed for growth in soft agar and reduced number of colonies and colony size was evident. FIG. 13 shows HT29 transduced with siRNA ZNF306 or vector only [pSUPER]. Cells were selected with puromycin (6 μg/ml) for 1 week. Resistant cells (5,000) were analyzed for growth in soft agar. Photomicrographs are taken 2 weeks later. ZNF306 was driving tumorigenecity and/or progression. siRNA targeting this transcription factor reduced growth in semi-solid medium as well as diminished the size of tumors formed orthotopically.

### Example 11

### ZNF306 Increases Resistance to 5-Fluorouracil

Since one of the hallmarks of colon cancer progression is acquired resistance to 5-Fluorouracil (5FU), it was determined whether ZNF306 over-expression conferred resistance to this drug. HCT116 cells expressing the empty vector or a pool of G418-resistant HCT116 cells overexpressing the ZNF306 cDNA were grown with 5FU concentrations used in vivo for 6 days and living cells enumerated. FIG. 14 shows the results of treatment of HCT116 cells expressing empty vector or ZNF306 cDNA, with the indicated 5-fluorouracil concentrations. Viable cells were counted 6 days later. It is evident that ZNF306 over-expression increases the resistance to this chemotherapeutic agent (FIG. 14). Thus, these data suggest that ZNF306 contributes to colon cancer progression.

### Example 12

### An Antibody Against the Endogenous ZNF306 Protein

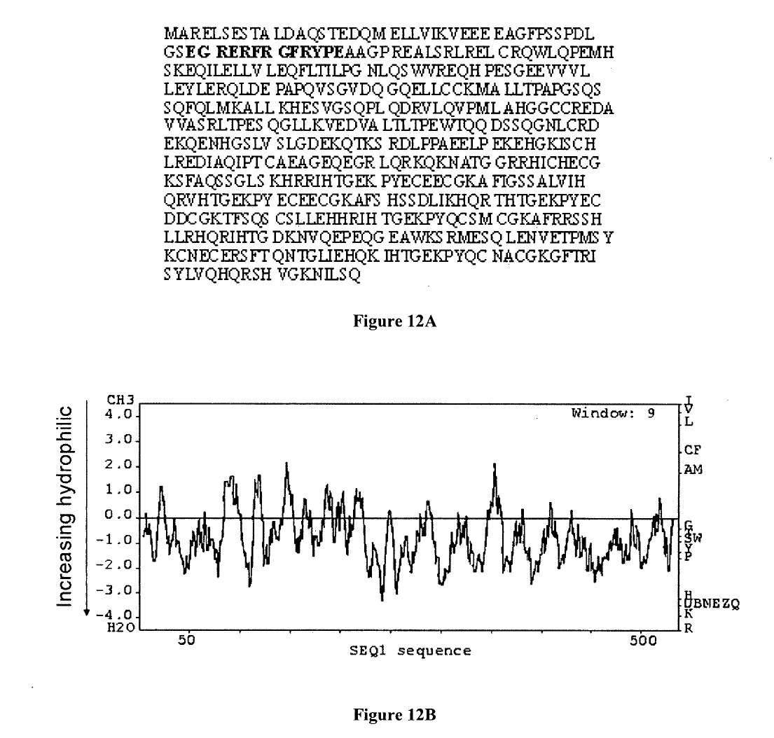

A peptide sequence (FIG. 12A, EGRERFRGFRYPE (SEQ ID NO:8), See Table 4 for abbreviations) has been identified suitable as immunogen based on the following criteria (a) its hydrophillicity (FIG. 12 B) and (b) its unique sequence as determined by a BLAST search. This peptide was KLH-carboxy-terminus conjugated by Sigma Genosys (The Woodlands, Tex.), 100-200 μg mixed with Freund's Adjuvant and injected into duplicate New Zealand White rabbits bi-weekly over a 10 week period. Serum was drawn after the 7th week and every other week thereafter.

The serum, and as a control, serum drawn from pre-immune rabbits, was tested for its reactivity (1:500 and 1:100 dilutions) with the FLAG-tagged ZNF306 (1-50 ng) in Western blotting. Success of antibody generation was defined by the fulfillment of two criteria. We sought to determine: first, whether we detect a 60 kDa band in the Western blots (run under reducing or non-reducing conditions) using the anti-serum derived from immunized rabbits but not pre-immune serum, and second, whether the 60 kDa band abolished when the anti-serum was pre-adsorbed with an excess of the immunizing peptide.

We detected the Flag-tagged ZNF306, titration studies (ranging from 1:10,000, to 1:100) will be performed to determine the optimal anti-serum dilution for detection of the ZNF306. This was carried out for all bleeds to determine the optimal dilution to use in Western blotting as well as the bleed corresponding to the peak titer. The bleed giving the best titer was determined, rabbits sacrificed and exsanguinated retaining the anti-ZNF306 anti-serum.

For specificity studies, whole cell extract (using RKO or HCT116 cells which express the endogenous ZNF306 transcript) was subjected to Western blotting with the anti-serum using a dilution identified in the previous experiments. We determined whether we can detect the endogenous ZNF306 protein. We detect a single band of the predicted size (60 kDa).

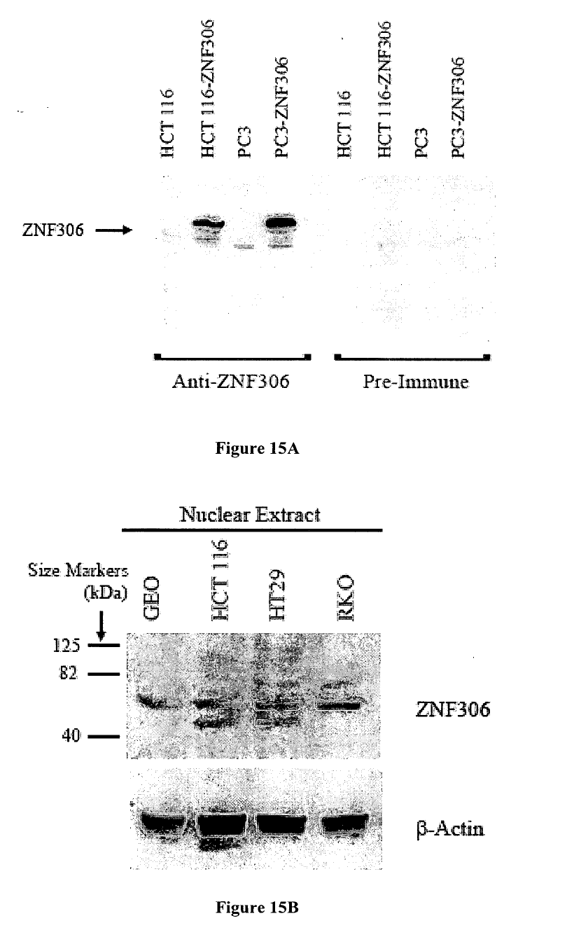

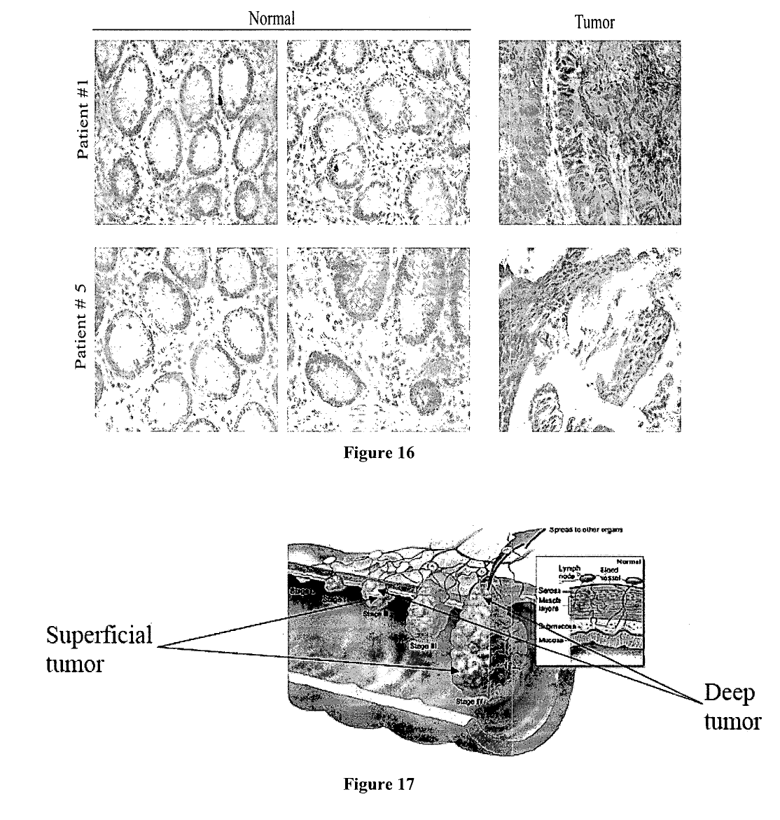

The results of Western blotting using the antibody can be seen in FIG. 15A. FIG. 15B demonstrates the same as FIG. 15A with the exception that 4 parental colon cancer cell lines were compared for endogenous ZNF306 protein. Note that the exposure in FIG. 15B is longer than FIG. 15A to reveal the endogenous protein. Immunohistochemistry showing reactivity (brown color) most pronounced in the tumor can be seen in FIG. 16. A 1:2000 dilution of the ZNF306 antiserum was used. DAB was used to visualize immunoreactivity.

### Example 13

### Immunohistochemistry Studies on Stage II and IV Tissues

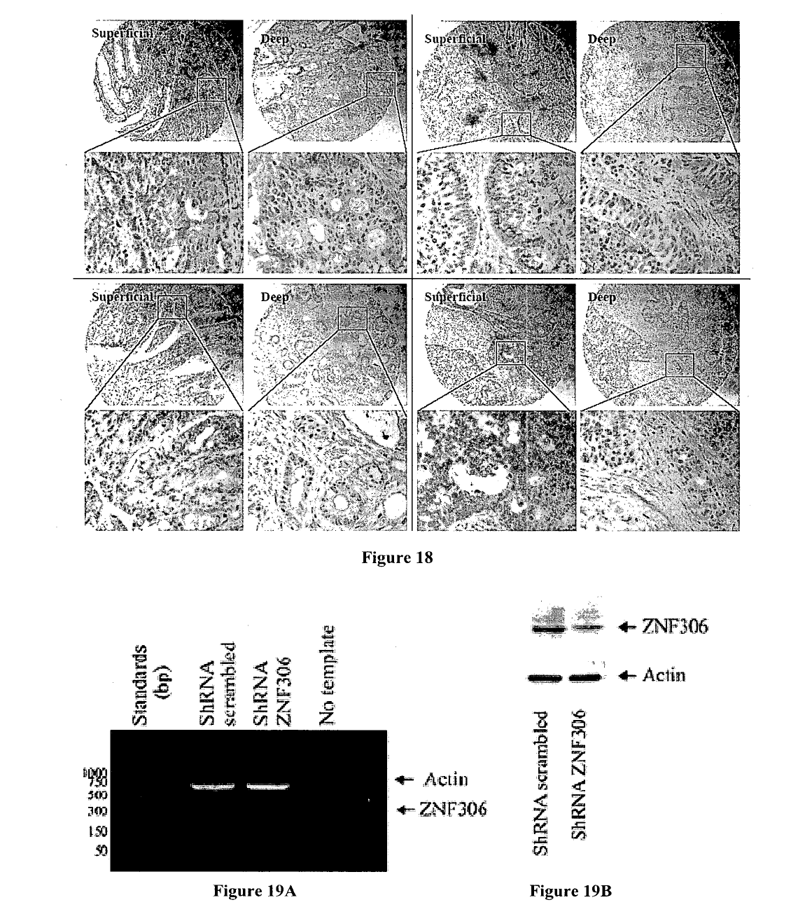

Immunohistochemistry on a colorectal tissue microarray of stage IV and II tissues (FIG. 17) was performed. Non-malignant mucosa and adenomatous tissue showed diminished reactivity with the anti-ZNF306 antibody. Of the 11 patients per stage, 8 and 11 showed tumor cell ZNF306-positive nuclei for Stage II and IV disease respectively. Interestingly, pronounced ZNF306 immunoreactivity was evident in tumor cell nuclei (arrows) of deeply invasive cancers but less so in matched superficial tumors. (FIG. 18.) Histomorphometric analysis indicated 78±17 and 14±11% (average±SD) ZNF306-positive nuclei for the deeply invasive and superficial tumor cells respectively a statistically significant (p<0.0001) difference. Further, stage IV tumor cells showed a greater % of ZNF306-positive nuclei compared with stage II cancers (52±18 and 9±10 respectively; p<0.0001). Some staining was also evident in inflammatory cells.

### Example 14

### ZNF306 Knockdown Modulates Colon Cancer Tumorigenecity

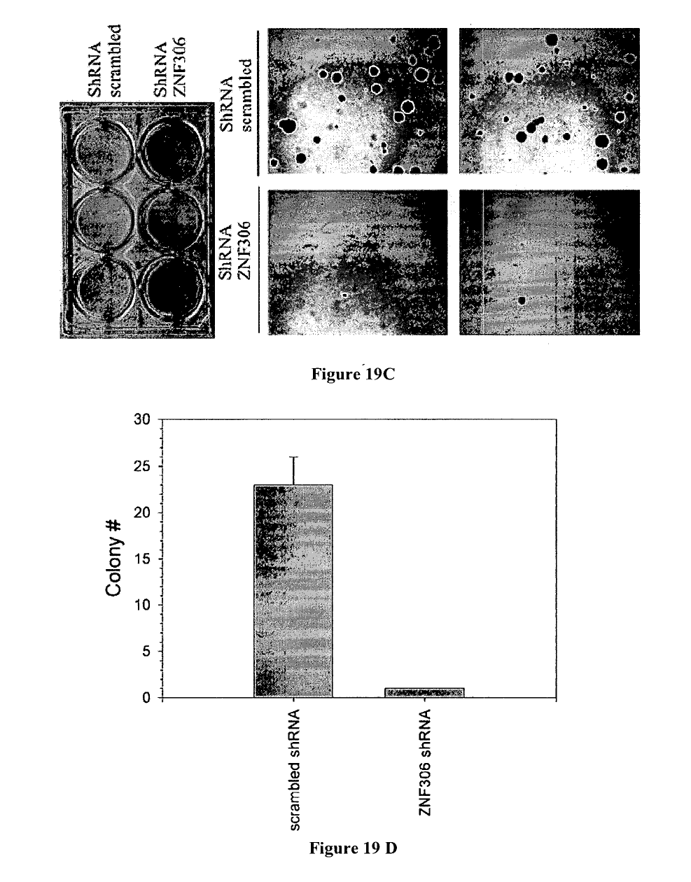

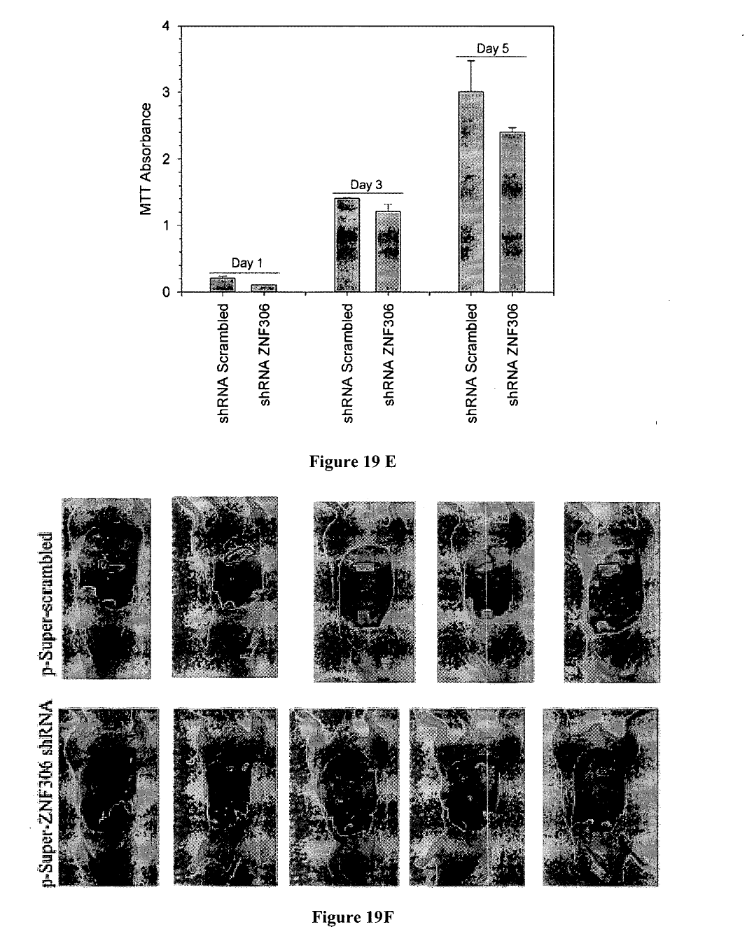

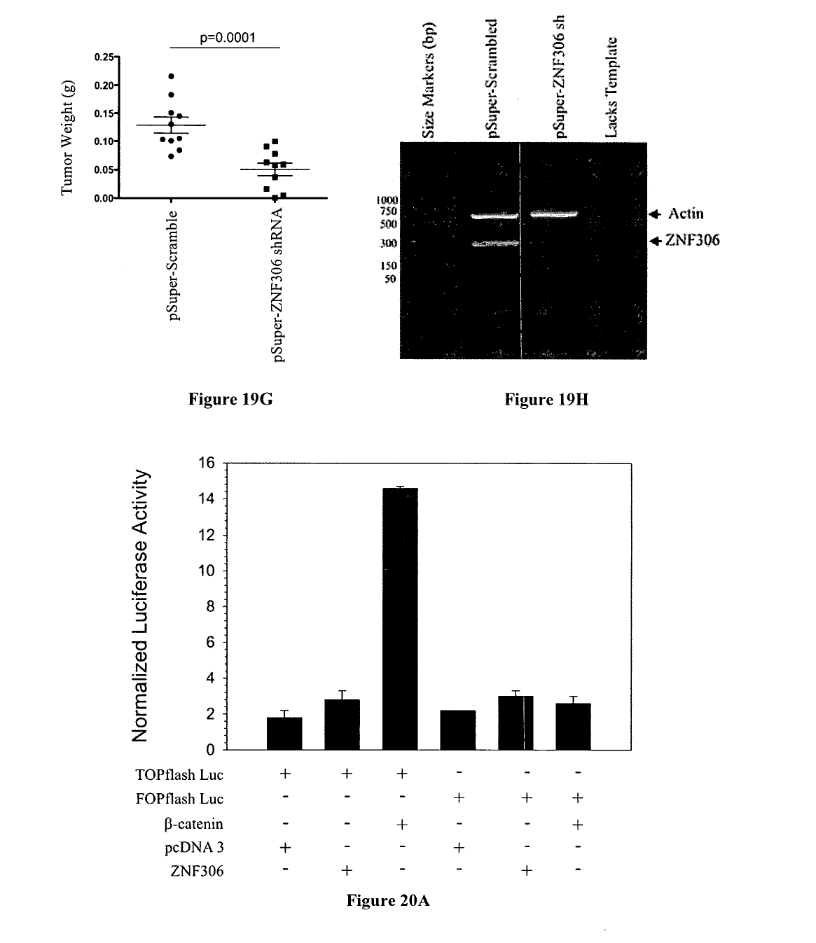

The effect of silencing ZNF306 on tumorigenecity was determined. First, ZNF306 was knocked down in RKO colon cancer cells showing the highest ZNF306 expression (FIG. 15B) and wild type for p53, APC, b-catenin, K-Ras, MADH4 and bearing a wild type allele for the PI3K catalytic domain (http://www.sanger.ac.uk/perl-/genetics/CGP/). RKO cells were transduced with a retro-virus encoding a ZNF306-targeting shRNA, or the scrambled sequence, and approximately 70% knockdown of endogenous ZNF306 was evident by RT-PCR and Western blotting (FIGS. 19A, B). Strikingly, ZNF306 repression markedly reduced anchorage-independent growth (FIGS. 19C, D). Note the yellow color of the pH indicator suggesting robust growth (anaerobic conditions) with scrambled shRNA-expressing cells in contrast to the orange color (aerobic conditions) with the ZNF306-knocked down cultures (FIG. 19C). Reduced colony number unlikely reflected slower monolayer proliferation (FIG. 19E). To corroborate the in vitro data, nude mice were injected orthotopically with RKO cells transduced with a ZNF306-targeting shRNA or the scrambled sequence. Intra-cecally injected RKO cells transduced to express the scrambled shRNA were highly tumorigenic (tumors circumscribed with solid line) whereas the cells knocked down for ZNF306 showed dramatically smaller tumors (FIGS. 19F and G). RT-PCR confirmed ZNF306 transcript knockdown in pooled tumor tissue from mice injected with the ZNF306-silencing vector (FIG. 19H).

### Example 15

### ZNF306 Does Not Stimulate p53, Tcf/Lef and TGF-β Responsive Reporters

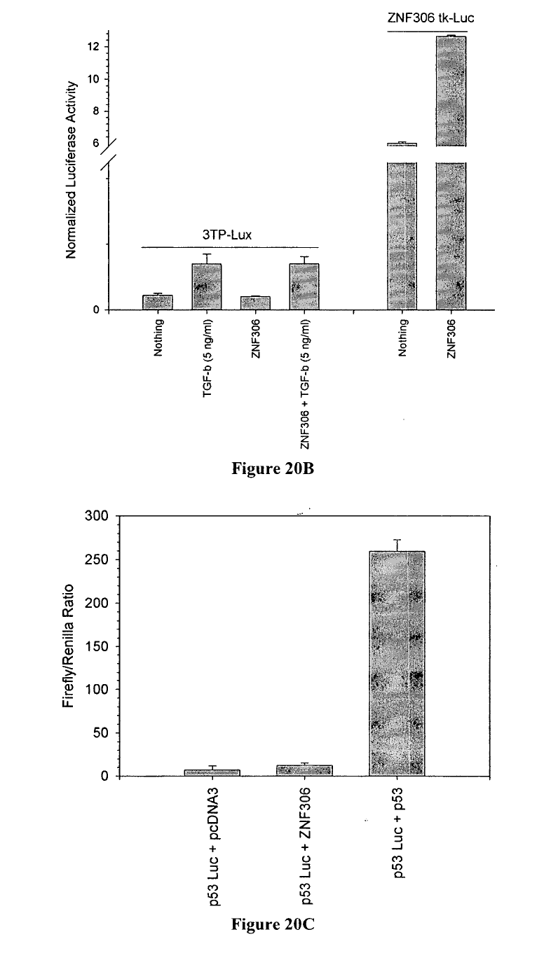

It was then determined if ZNF306 intersects with p53, Wnt or TGF-β pathways, all implicated in sporadic colorectal cancer development/progression, by transiently co-transfecting colon cancer cells with pathway-responsive reporters and a ZNF306 expression construct. In RKO cells, wild type for APC and β-catenin, ZNF306 failed to activate the Wnt pathway-responsive TOPflash reporter whereas the positive control β-catenin) caused a robust induction (FIG. 20A). Similarly, while TGF-β treatment induced a TGF-β-responsive promoter (3TP-Lux) in FET colon cancer cells (FIG. 20B), ZNF306 expression failed to activate this reporter although it was effective (FIG. 20B) on an artificial promoter comprised of tandem ZNF306 binding motifs. Finally, ZNF306 expression had minimal effect on a p53 reporter (FIG. 20C) in p53-wt RKO cells whereas a p53 expression construct activated this reporter 20 fold.

### Example 16

### ZNF306 is Also Expressed in Colorectal Tumor Cells Quiescent for the Wnt Pathway and Wild Typefor K-Ras and p53

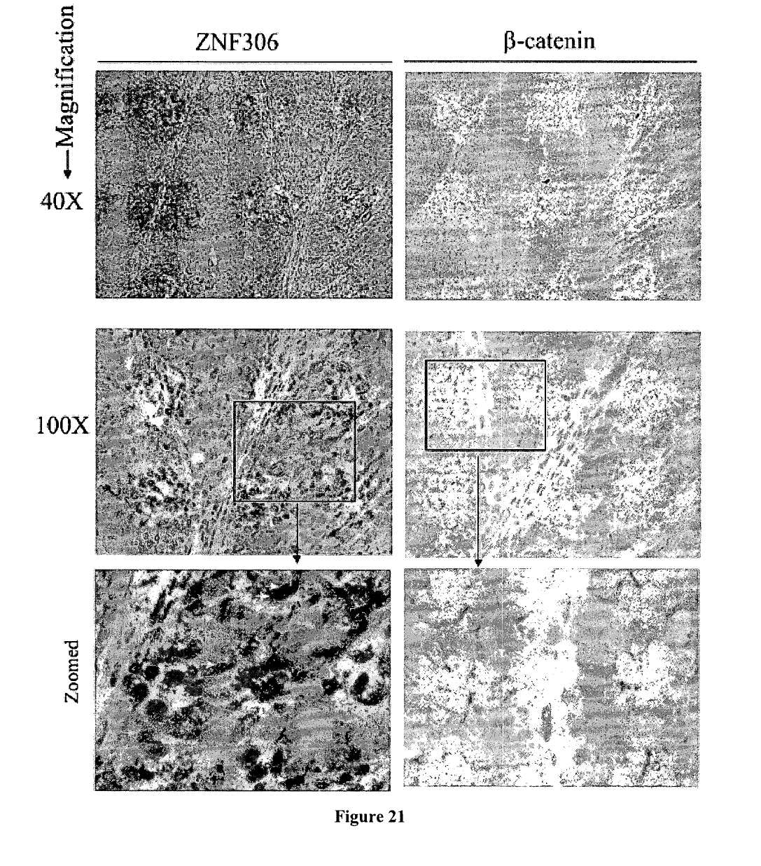

Since we were intrigued with the possibility that ZNF306 contributes to tumor progression in colorectal cancers wild type for some of the commonly activated genes, ZNF306 expression was determined (FIG. 21) in sections from tumors genotyped as concurrently wild type for APC, K-Ras and p53. To confirm a quiescent Wnt pathway, serial sections were stained for β-catenin. Of 5 patients, 4 showed non-nuclear β-catenin (confirming a silent Wnt pathway) concurrent with pronounced nuclear ZNF306 (FIG. 21, arrows). Thus, ZNF306 is also expressed in colorectal tumor cells quiescent for the Wnt pathway and wild type for K-Ras and p53.

### Example 17

### Integrin β4 is a Downstream Effector of ZNF306

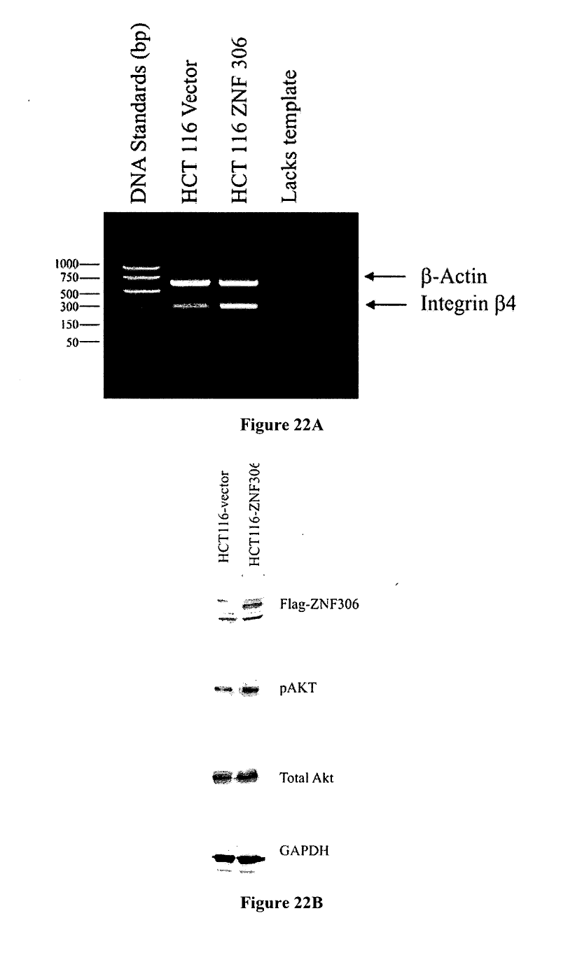

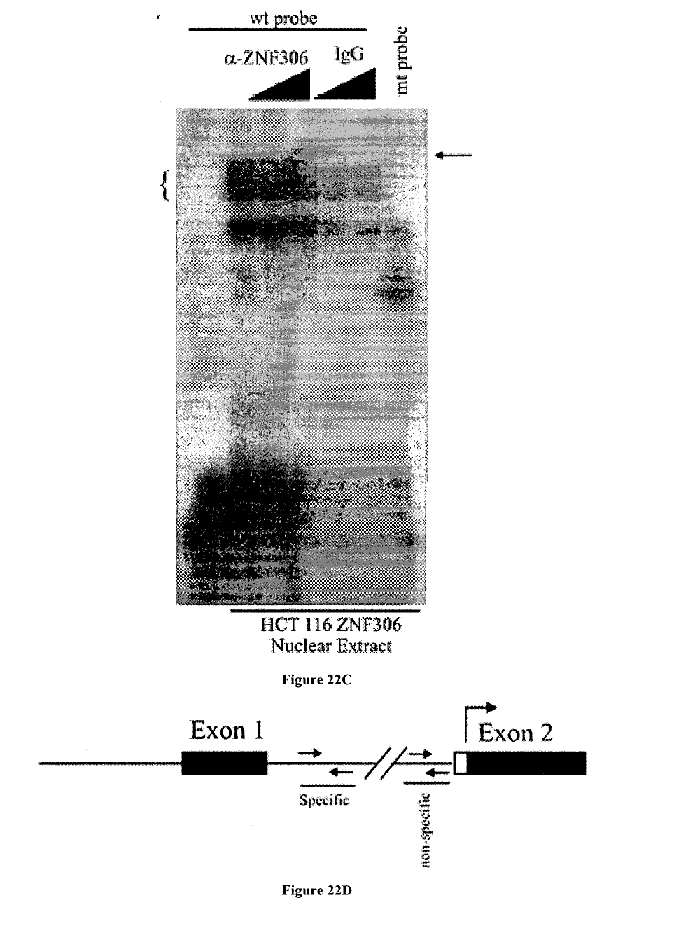

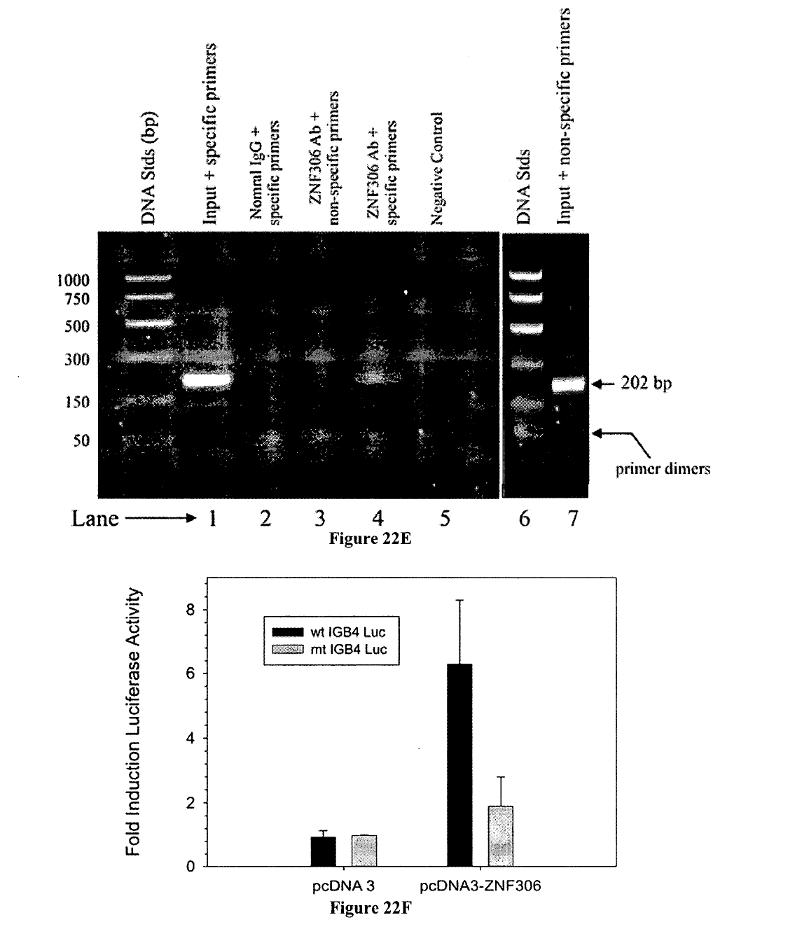

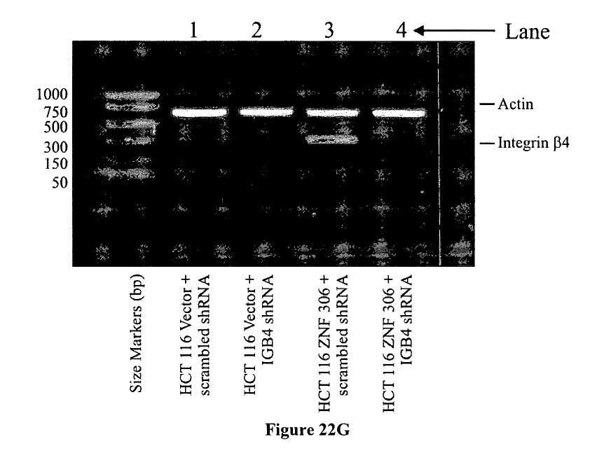

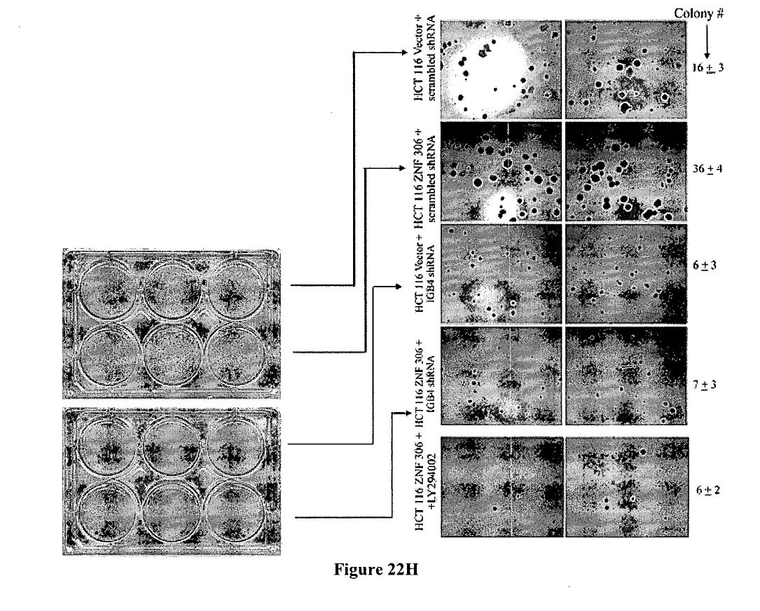

The integrin β4 induction in expression profiling was of particular interest since this cell surface protein has recently been implicated in mammary tumorigenecity, tumor cell migration, and its expression is up-regulated in colorectal cancer. Moreover, integrin β4 stimulates the PI3K signaling module 29 functioning in colorectal cancer progression 24. RT-PCR showing elevated integrin β4 mRNA in pooled tumors generated with ZNF306-overexpressing HCT116 cells (FIG. 22A) validated the expression profiling data. Note that HCT116 cells express wild type integrin β4. Further, increased phosphorylated Akt levels (FIG. 22B), indicative of activated PI3K signaling, was evident in the ZNF306-overexpressing HCT116 cells consistent with integrin β34 converging on this module.

If integrin β4 is a direct ZNF306 target, the regulatory region bearing the binding motif identified by CAST-ing would be predicted to be ZNF306-bound. The first intron, regulatory for gene expression, included a putative ZNF306 binding site (TGAGGGG) (SEQ ID NO:9) conforming to the KRDGGGG consensus site, where K is G/T, R is A/G, and D is A/G/T, and we determined the role of this element in ZNF306-dependent regulation of integrin β4. In EMSA, an oligonucleotide spanning this binding site (wt probe), but not one substituted at the core sequence (mt probe), produced a retarded band (FIG. 22C, parenthesis) with nuclear extract from ZNF306 cDNA-expressing HCT116 cells. The retarded band was “supershifted” (arrow) with our anti-ZNF306 antibody but not by an equivalent amount of pre-immune IgG. Moreover, in chromatin immunoprecipitation assays (FIG. 22E), the anti-ZNF306 antibody in conjunction with primers (specific) flanking the ZNF306-binding motif (FIG. 22D) generated a band (FIG. 22E, lane 4) whereas no band was evident with normal IgG (lane 2) or primers (non-specific) located 1821 bp downstream from the ZNF306 recognition site (lane 3). Next, we determined if this ZNF306 binding element was regulatory for expression. Duplicate tandem copies of the integrin β4-derived motif or the element substituted to prevent ZNF306 binding (see FIG. 22C) was fused upstream of a minimal tk promoter-luciferase construct. Co-transfection of a ZNF306 expression plasmid with the reporter driven by the wild type ZNF306 motif (wt IGB4 Luc) induced luciferase activity 6 fold compared with the empty expression construct (FIG. 22F) whereas substitution of the motif to abrogate ZNF306 binding (mt IGB4 Luc) nearly abolished this stimulation. Thus, integrin β4 is probably a direct downstream target of ZNF306.

To determine if integrin β4 is a ZNF306 effector, pooled HCT116 cells expressing a ZNF306 cDNA or the empty vector were transduced with a retrovirus bearing a integrin β4-targeting shRNA. Expectedly, while ZNF306 induced integrin β4 mRNA levels (FIG. 22G, compare lanes 3 and 1), the integrin β4-targeting shRNA practically ablated integrin β4 transcript levels for both HCT116 cells expressing the ZNF306 and the corresponding empty vector, (FIG. 22G lanes 2 and 4). Strikingly, integrin β4 knockdown countered the ZNF306-dependent augmentation of anchorage-independent growth (p<0.0001) as did a PI3K inhibitor (L Y294002) (FIG. 22H). The ability of the integrin β4-targeting shRNA to reduce colony growth (p=0.0001) in HCT116 cells lacking the ZNF306 cDNA probably reflects endogenous ZNF306 silencing (compare FIG. 22G lanes 1,2). The integrin P4-targeting shRNA only marginally reduced monolayer growth (data not shown). Thus, these data implicate integrin P4 as a down-stream effector of ZNF306.

### Example 18

### Liposomal Delivery of siRNA Targeting ZNF306 has an in Vivo-Effect on Tumor Growth

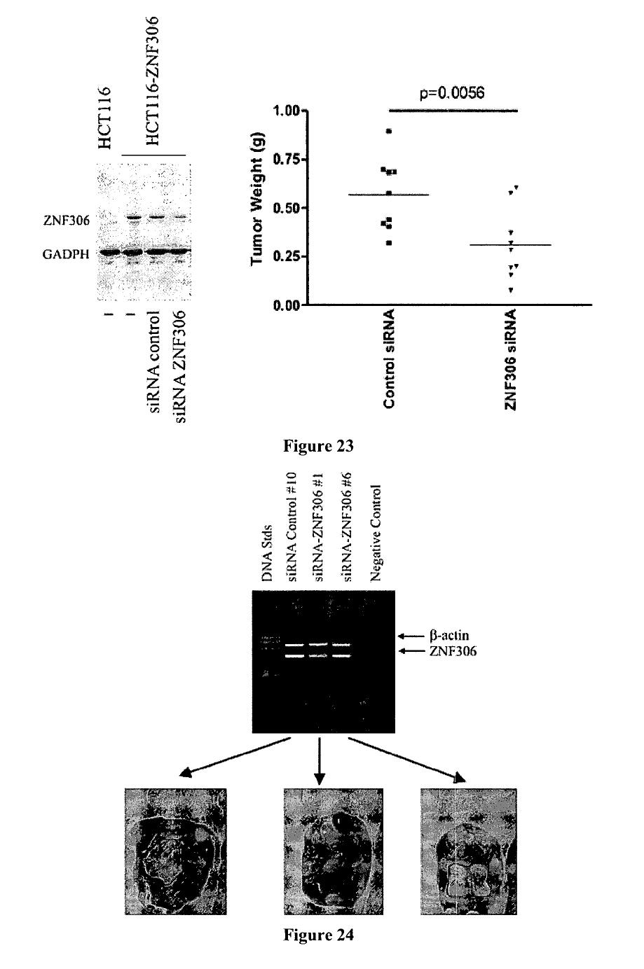

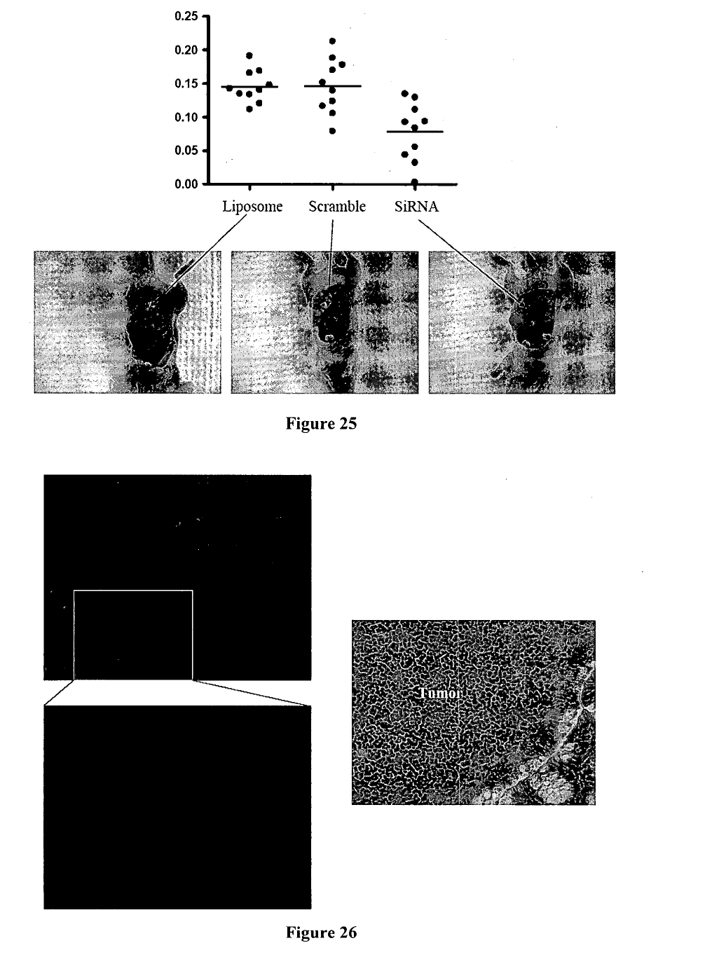

Intravenous (IV) delivery of siRNA incorporated into neutral liposomes allows efficient delivery to tumor tissue, and has therapeutic efficacy in preclinical proof-of-concept studies using EphA2-targeting siRNA (Landen et al., Cancer Research 65, 6910-6918, 2005). Thus, ZNF306 SiRNA was incorporated into the neutral liposome 1,2-dioleoyl-sn-glycero-3-phosphatidylcholine (DOPC). Male athymic nude mice (NCr-nu) were used to establish orthotopic colon tumor with HCT116-ZNF306 stable cells or RKO cells. Therapy began 1 week after tumor cell injection. SiRNA (nonspecific or ZNF406 targeting, 150 Dg/kg) in liposomes, or empty liposomes, were injected twice weekly i.v. in 150 to 200 DL volume (depending on mouse weight) with normal pressure. Mice (n=10 per group) were monitored for adverse effects, and tumors were harvested after 7 weeks (HCT116-ZNF306 tumors) or 5 weeks (RKO tumors) of therapy or when any of the mice began to appear moribund. Mouse weight, tumor weight, and distribution of tumor were recorded. Vital organs were also harvested and necropsies were done by a board-certified pathologist for evidence of tissue toxicity. As shown in supplementary FIGS. 23, 24, and 25, treatment with anti-ZNF306 siRNA was effective in reducing tumor weight, leading to 86% reduction compared with treatment with control siRNA alone. Interestingly, RT-PCR revealed that some larger tumor in ZNF306-SiRNA treatment group did not show reduction of ZNF306 mRNA, while small tumor had dramatically decreased ZNF306 mRNA expression, further demonstrated the specificity and effectiveness of targeting ZNF306 therapy.

Studies to determine uptake of single-dose fluorescent siRNA in tissue or silencing potential of siRNA against ZNF306 were initiated about 5 weeks after injection. In these mice, liposomal siRNA (dose 150 Dg/kg) was given twice a week, and tumors were harvested 24 hours after the last dose. Tissue specimens were frozen in OCT medium for frozen slide preparation. Frozen sections were cut at 8 mm sections, fixed with acetone, exposed to 1.0 mg/mL Hoescht (Molecular Probes, in PBS) for 10 minutes to stain nuclei, washed, and covered with propylgallate and cover slips for microscopic evaluation. Conventional microscopy (FIG. 26) was done with a Zeiss AxioPlan 2 microscope (Carl Zeiss, Inc, Germany), Hamamatsu ORCA-ER Digital camera (Hamamatsu Corp, Japan), ImagePro software (Media Cybernetics, Silver Spring, Md.).

TABLE 4
Amino Acid Abbreviations

| Amino Acid | Abbreviation | Symbol |
|---|---|---|
| Glycine | Gly | G |
| Alanine | Ala | A |
| Proline | Pro | P |
| Valine | Val | V |
| Leucine | Leu | L |
| Isoleucine | Ile | I |
| Methionine | Met | M |
| Phenylalanine | Phe | F |
| Tyrosine | Tyr | Y |
| Tryptophan | Trp | W |
| Serine | Ser | S |
| Threonine | Thr | T |
| Cysteine | Cys | C |
| Asparagine | Asn | N |
| Glutamine | Gln | Q |
| Lysine | Lys | K |
| Histidine | His | H |
| Arginine | Arg | R |
| Aspartate | Asp | D |
| Glutamate | Glu | E |

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A composition comprising a siNA component that binds to a nucleotide sequence encoding ZNF306 protein and a lipid component.

2. The composition of claim 1, wherein the siNA prevents translation of a gene transcript that promotes growth of a cancerous or pre-cancerous mammalian cell.

3. The composition of claim 1, wherein the siNA has a target sequence of 5′-UAUCGUGCCACCUGAGAGA-3′.

4. The composition of claim 1, wherein the sequence of the siNA is 5′-AAUUCUCCGAACGUGUCACGU-3′.

5. The composition of claim 1, wherein the lipid component is in the form of a liposome.

6. The composition of claim 1, wherein the siNA is encapsulated in the liposome.

7. The composition of claim 1, wherein the siNA is siRNA.

8. The composition of claim 1, wherein the composition is comprised in a pharmaceutically acceptable carrier.

9. The composition of claim 8, wherein the pharmaceutically acceptable carrier is formulated for administration to a human.

10. The composition of claim 1, wherein the lipid component has an essentially neutral charge.

11. The composition of claim 2, wherein the siNA prevents the expression of an oncogene transcript.

12. The composition of claim 1, wherein the composition further comprises a chemotherapeutic.

13. The composition of claim 12, wherein the lipid component is in the form of a liposome and the chemotherapeutic is encapsulated within the liposome.

14. The composition of claim 11, wherein the siNA is a siRNA and the siRNA is encapsulated within the liposome.

15. An antibody comprising a human constant region that binds to at least a portion of a ZNF306 protein.

16. An antibody as defined in claim 15, which is chimeric.

17. An antibody as defined in claim 15, which is humanly acceptable.

18. An antibody as defined in claim 15, which is conjugated to an anti-tumor agent.

19. An antibody as defined in claim 15, which is a monoclonal antibody.

20. An antibody, as defined in claim 15, wherein the antibody is specific for at least a portion of the peptide sequence EGRERFRGFRYPE.

21. A method for diagnosing or staging or monitoring cancer, the method comprising determining the level of ZNF306 protein on a cancer cell present in a tissue suspected of being positive for cancer and comparing the level to the level of ZNF306 protein on normal tissue, whereby an increase in the level of ZNF306 protein in the suspect tissue over the level of ZNF306 protein in the normal tissue indicates the stage of cancer in the suspect tissue.

22. The method of claim 21 wherein the cancer originated in the bladder, blood, bone, bone marrow, brain, breast, rectum, ovarian, esophagus, gastrointestine, gum, head, kidney, liver, lung, nasopharynx, neck, prostate, skin, stomach, testis, tongue, or uterus.

23. The method of claim 21 wherein the cancer is colon cancer.

24. A method as defined in claim 21, wherein said level of the ZNF306 protein is determined by exposing the suspect and the normal tissues a ZNF306 protein binding antibody and comparing the amount of antibody bound by each tissue, wherein an increase in the level of bound antibody by the suspect tissue over the level of bound antibody by the normal tissue indicates the stage of cancer in the suspect tissue.

25. The method of claim 24 wherein the antibody is specific for at least a portion of the peptide sequence EGRERFRGFRYPE.

26. A method for screening a compound that inhibits or prevents cancer cell proliferation, the method comprising determining a first amount of ZNF306 protein expressed by cancer cells exposed to the compound, wherein the cancer cells overexpress ZNF306 protein; and comparing the first amount of ZNF306 protein to a second amount of ZNF306 protein expressed by the cancer cells that have not been exposed to the compound; whereby the first amount being less than the second amount indicates that the compound may inhibit or prevent ZNF306 cancer cell proliferation.

27. A kit for diagnosing or staging cancer, the kit comprising an antibody as defined in any of claims 15-20.

28. A kit for screening compounds that inhibit or prevent cancer cell proliferation, the kit comprising an antibody as defined in any of claims 15-20.

29. A method of preventing growth of a cancerous or precancerous mammalian cell comprising administering to the cell a composition a siNA component that binds to a nucleotide sequence encoding ZNF306 protein and a lipid component, wherein the siNA prevents translation of a gene transcript that promotes growth of the cancerous or precancerous mammalian cell.

30. The method of claim 29, wherein the siNA has a target sequence of 5′-UAUCGUGCCACCUGAGAGA-3′.

31. The method of claim 29, wherein the sequence of the siNA is 5′-AAUUCUCCGAACGUGUCACGU-3′.

32. A method of treating cancer comprising administering to a mammal a composition comprising a siNA component that binds to a nucleotide sequence encoding ZNF306 protein and a lipid component.

33. The method of claim 32, wherein the siNA has a target sequence of 5′-UAUCGUGCCACCUGAGAGA-3′.

34. The method of claim 32, wherein the sequence of the siNA is 5′-AAUUCUCCGAACGUGUCACGU-3′.

35. The method of claim 32 wherein the cancer originated in the bladder, blood, bone, bone marrow, brain, breast, rectum, ovarian, esophagus, gastrointestine, gum, head, kidney, liver, lung, nasopharynx, neck, prostate, skin, stomach, testis, tongue, or uterus.

36. The method of claim 32 wherein the cancer is colon cancer.


SEQUENCE LISTING (9 sequences)

SEQ ID NO: 1 - RNA, 21 nt, Artificial, Natural, or Synthetic Sequence
  siRNA sequence
aauucuccgaacgugucacgu

SEQ ID NO: 2 - RNA, 19 nt, Artificial, Natural, or Synthetic Sequence
  siRNA targeting sequence
uaucgugccaccugagaga

SEQ ID NO: 3 - DNA, 75 bp, Artificial, Natural, or Synthetic Sequence
  Random oligonucleotide
cacgtgagttcagcggatcctgtcgnnnnnnnnnnnnnnnnnnnnnnnnngaggcgaattcagtgcaactgcagc

SEQ ID NO: 4 - DNA, 18 bp, Artificial, Natural, or Synthetic Sequence
  primer
ggccctgaccctcacccc

SEQ ID NO: 5 - DNA, 20 bp, Artificial, Natural, or Synthetic Sequence
  primer
cagatgtgccgcctccctcc

SEQ ID NO: 6 - DNA, 21 bp, Artificial, Natural, or Synthetic Sequence
  primer
gacgatgacgacaagggatcc

SEQ ID NO: 7 - DNA, 20 bp, Artificial, Natural, or Synthetic Sequence
  primer
ccagcagctccaggatctgc

SEQ ID NO: 8 - protein, 13 aa, Artificial, Natural, or Synthetic Sequence
  peptide from ZNF306 protein sequence
EGRERFRGFRYPE

SEQ ID NO: 9 - DNA, 7 bp, Artificial, Natural, or Synthetic Sequence
  binding site
tgagggg